(19) United States
(12) Patent Application Publication
(10) Pub. No.: US 2025/0288824 A1
(43) Pub. Date: Sep. 18, 2025

(54) SUPPORTABLE PHOTOBIOMODULATION DEVICES

(71) Applicant: GembaRed LLC, Glastonbury, CT (US)

(72) Inventor: Andrew LaTour, Lebanon, CT (US)

(73) Assignee: GembaRed LLC, Glastonbury, CT (US)

(21) Appl. No.: 18/605,142

(22) Filed: Mar. 14, 2024

(51) Int. Cl. A61N 5/06

(52) U.S. Cl. CPC A61N 5/062; A61N 2005/0633; A61N 2005/064; A61N 2005/0652; A61N 2005/0659; A61N 2005/0663

(57) ABSTRACT

Disclosed is a light panel including: a housing having: a face side including a face height and a face width, a first side and a second side at opposite ends of the face width, and a top side and a bottom side at opposite ends of the face height; a plurality of light emitting diodes configured to transmit electromagnetic radiation from the face side; a leg having a proximal end and a distal end; a hinge rotatably connecting the proximal end to the housing; and a stability bar on the support leg, wherein the support leg is rotatable between a closed position, and an open position where the distal end is farther from the housing than the proximal end and the light panel is supported on a surface in an upright position with a weight of the light panel being distributed between the leg and the housing.

### BACKGROUND OF THE DISCLOSURE

Light therapy is a method of providing directed light to a user in order to produce therapeutic benefits in the user. For example, light therapy can involve exposing a user to a lighting device designed to provide beneficial electromagnetic radiation spectra to the user. Such radiation can affect metabolic function of the user in a beneficial way. Light therapy lighting devices can include handheld lighting devices, wearable lighting devices, and larger lighting devices such as light panels. Light panels can offer a larger potential light dose to a user by outputting a greater amount of electromagnetic radiation. However, in order for the dose potential to be realized the light output by the light panel needs to reach the target area (e.g., the user's body).

### BRIEF DESCRIPTION OF THE DRAWINGS

The foregoing and other objects, features, and advantages of the disclosure will be apparent from the following description of particular implementations of the disclosure, as illustrated in the accompanying drawings in which like reference characters refer to the same parts throughout the different views. The drawings are not necessarily to scale, emphasis instead being placed upon illustrating the principles of the disclosure.

FIG. 1 is a schematic illustration including a front view (a) and a side view (b) of a light panel having a support leg and stability bar.

FIG. 2 is a schematic illustration including a front view (a) and a side view (b) of a support leg and stability bar.

FIG. 3 is a schematic illustration of a front view of a light panel support stand having multiple light panels mounted thereto.

FIG. 4 is a schematic illustration of a back view of the light panel support stand of FIG. 3.

FIG. 5 is a schematic illustration including a front view (a) and a side view (b) of a light panel support stand.

FIG. 6 is a schematic illustration of a top view of a light panel support stand having multiple light panels mounted thereto.

FIG. 7 is a schematic illustration including a front view (a) and a back view (b) of a light panel support stand having multiple light panels mounted thereto by a two-section support arm/bracket.

FIG. 8 is a schematic illustration including a front view (a) and a back view (b) of a light panel support stand having multiple light panels mounted thereto by an upright frame member and a support arm/bracket.

FIG. 9 is a schematic illustration of a top view (a) and a back view (b) of a light panel support stand having multiple light panels mounted thereto which are angled by spacers.

FIG. 10 is a schematic illustration including a front view (a) and a back view (b) of a light panel support stand having multiple light panels mounted thereto by a two-section support arm/bracket.

### DETAILED DESCRIPTION

The present disclosure relates to therapeutic lighting devices and more particularly to methods and devices for supporting therapeutic lighting devices to target a user of the therapeutic light.

It has been recognized that a light panel can be difficult for a user to move or arrange in a beneficial way so as to target their body with the light transmitted from the light panel. For example, users of body sized panels can be forced to resort to expensive frames which can be difficult to maneuver in a user's home, door or wall hanging systems which can interfere with use of the door or wall and/or can leave the panel vulnerable to damage (e.g., when not used), or leaning the panel against a wall or object which can interfere with use of the wall or object and/or leave the panel vulnerable to damage. Additionally, operation of a therapeutic lighting device requires electricity to run the light. A user is unable to utilize light that does not reach the user's body (e.g., light which bypasses the user) which can result in inefficiency of the light therapy treatment (e.g., where an efficiency of the treatment can be defined as light absorbed divided by light supplied from the device or power supplied to the device).

One aspect of the present disclosure is a system including a therapeutic light panel having a stand mechanism for supporting the panel. The stand mechanism can be built into and/or onto the panel to allow for quick and easy setup of the panel when a user is seeking therapy and quick and easy removal of the panel after therapy is complete. The stand mechanism can include an angling device so that light from the panel can be angled to provide the greatest coverage to the user's body thereby improving the efficiency of the treatment.

In an implementation of the system, the stand mechanism can include a support leg attached to the housing of the light panel. The support leg can be attached to the panel in any way so as to provide a support onto which the panel's weight can be distributed. For example, the support leg can be attached to the light panel on a rear surface (e.g., opposite from the side which light is transmitted from the light panel), a side surface, a top surface, a bottom surface of the panel, or even the front surface (e.g., the side from which light is transmitted from the light panel).

In an implementation of the system, the stand can include a support leg having a stability bar, or equivalent support mechanism, to enhance the stability of the stand when the panel's weight is distributed to the support leg. For example, the stability bar can be configured to extend laterally from the support leg to provide lateral support to the stand to reduce or prevent the incidence of lateral tipping of the supported light panel by spreading the footprint of the supported light panel and/or lowering the center of mass of the supported light panel thereby increasing a force required to induce lateral tipping of the supported light panel.

Another aspect of the present disclosure is a system including an adjustable frame for mounting one or more light panels for directing therapeutic light to a user. The adjustable frame can include one or more angling devices for angling the one or more panels in a way most beneficial to a user, e.g., to improve dose efficiency, such as by directing a greater portion of therapy light toward the user's body in comparison to other methods (e.g., leaning a panel against a wall or hanging the panel from a door). The adjustable frame can include mounting brackets which can provide easy assembly of the one or more panels onto the adjustable frame.

In an implementation of the system, the adjustable frame can include a supportive base with wheels to provide greater mobility to the system, e.g., allowing for easy movement of the system even after the one or more panels have been attached to the adjustable frame.

In an implementation of the system, the adjustable frame can include a support arm extending from an upright frame member, the support arm can be configured to hold a first light panel and the upright frame member can be configured to hold a second light panel. The support arm can be configured so that an angle between the support arm and the upright frame member (e.g., and panels thereon) can be adjusted to allow for light transmitted from the two light panels to intersect (e.g., such as at a target distance from the light panels where a user would receive light therapy treatment).

In an implementation of the system, the adjustable frame can include two support arms extending from an upright frame member, each one of the two support arms can be configured to hold a light panel and can be configured so that an angle between the two support arms (e.g., and panels thereon) can be adjusted to allow for light transmitted from the two light panels to intersect (e.g., such as at a target distance from the light panel where a user would receive treatment).

In an implementation of the system, the adjustable frame can include two support arms extending from an upright frame member, each one of the two support arms and the upright frame member can be configured to hold a light panel and can be configured so that a first angle between the upright frame member and a first support arm (e.g., and panels thereon), and a second angle between the upright frame member and a second support arm (e.g., and panels thereon), can be adjusted to allow for light transmitted from the three light panels to intersect (e.g., such as at a target distance from the light panel where a user would receive treatment).

In one aspect of the present disclosure a light panel can include: a housing having: a face side including a face height extending along a major dimension of the housing and a face width extending along a minor dimension of the housing, a first side and a second side at opposite ends of the face width, and a top side and a bottom side at opposite ends of the face height; a plurality of light emitting diodes configured to transmit electromagnetic radiation from the face side; a support leg having a proximal end and a distal end; a hinge rotatably connecting the proximal end to the housing; and a stability bar on the support leg, extending away from the support leg. The support leg can be rotatable between a closed position where both the proximal end and the distal end are adjacent the housing, and an open position where the distal end is farther from the housing than the proximal end and the light panel is supported on a floor surface in an upright position with a weight of the light panel being distributed between the support leg and the housing.

In an implementation, the housing can include a back side opposite the face side and the hinge is connected to the housing on the back side.

In an implementation, the hinge can include a first hinge and a second hinge, and the first hinge is connected to the first side and the second hinge is connected to the second side.

In an implementation, the support leg can include a first support leg connected to the first hinge and a second support leg connected to the second hinge.

In an implementation, the stability bar can include a friction surface configured to be contactable with the floor surface and prevent the stability bar from sliding on the floor surface with the light panel is in the upright position.

In another aspect of the present disclosure the light panel can further include: one or more feet connected to the stability bar. The friction surface can include one or more friction surfaces respectively corresponding to the one or more feet.

In an implementation, the stability bar can be fastened to the support leg by one or more fasteners.

In an implementation, the support leg can include two or more portions oppositely extended in the minor dimension beyond each side of the two sides of the housing to form the stability bar integral to the support leg.

In an implementation, the plurality of light emitting diodes can be configured to emit electromagnetic radiation having a wavelength of greater than or equal to about 400 nanometers and less than or equal to about 1400 nanometers.

In an implementation, wherein the support leg can have a support leg length of greater than or equal to one-half the face height of the light panel.

In an implementation, the support leg can include a plurality of support legs and each support leg of the plurality of support legs can have a length of greater than or equal to one-half the face height of the light panel.

In an implementation, the hinge can have an axis of rotation that is oriented substantially parallel to the floor surface with the light panel in the upright position.

In an implementation, the stability bar can be configured to counter-balance the light panel.

In an implementation, the stability bar can include a first stability bar and a second stability bar, the first stability bar can be on the support leg and can extend away from the support leg, and the second stability bar can be connected to the bottom side of the housing, can extend in the minor dimension beyond the first side of the housing and the second side of the housing, and can include a friction surface which can be configured to be contactable with the floor surface to prevent the second stability bar from sliding on the floor surface with the light panel is in the upright position.

In an implementation, one or more feet can be connected to the bottom side of the housing and can be interposed between the bottom side of the housing and the floor surface with the light panel in the upright position.

In an implementation, the hinge can connect the support leg on a back side of the housing adjacent to the bottom side of the housing and can be configured so that the distal end of the support leg rotates away from the top side of the housing when the support leg is rotated from the closed position to the open position, wherein in the open position the support leg is contactable with the floor surface and the weight of the light panel is distributed along a length of the support leg.

In one aspect of the present disclosure a light panel support stand can include: a base; an upright frame member connected to the base and configured to support a first light panel having a first face side; a first support arm connected to the upright frame member, and configured to support a second light panel having a second face side; and an angling device connected to at least one of the upright frame member or the first support arm, and, with the upright frame member supporting the first light panel and the first support arm supporting the second light panel, the angling device is configurable to form an angle between the first light panel and the second light panel so that light transmitted from the first face side intersects light transmitted from the second face side.

In an implementation, the angling device can be between the first support arm and the second light panel, can at least partially support the second light panel on the first support arm, and can form the angle.

In an implementation, the angling device can include a spacer configured to space at least one side of the second light panel from the first support arm to form the angle.

In an implementation, the angling device can include a hinge rotatably connecting the first support arm to the upright frame member so that the first support arm is rotatable relative to the upright frame member to form the angle.

In another aspect of the present disclosure the light panel support stand can further include: a first mounting plate attachable to the upright frame member to support the first light panel on the upright frame member.

In another aspect of the present disclosure the light panel support stand can further include: a first mounting plate attachable to the upright frame member to support the first light panel on the upright frame member; and a second mounting plate attachable to the first support arm to support the second light panel on the first support arm.

In another aspect of the present disclosure a light panel support stand can further include: a first mounting bracket attachable to the first light panel and having a first engagement configured to engage a first corresponding engagement on the first mounting plate to hold the first light panel on the first mounting plate; and a second mounting bracket attachable to the second light panel and having a second engagement configured to engage a second corresponding engagement on the second mounting plate to hold the second light panel on the second mounting plate.

In another aspect of the present disclosure a light panel support stand can further include: a first mounting bracket attachable to the first light panel and having a first engagement configured to engage a first corresponding engagement on the upright frame member to hold the first light panel on the upright frame member; and a second mounting bracket attachable to the second light panel and having a second engagement configured to engage a second corresponding engagement on the first support arm to hold the second light panel on the first support arm.

In one aspect of the present disclosure a light panel support stand can further include: a second support arm connected to the upright frame member, and configured to support a third light panel having a third face side. The angling device can include a first angling device and a second angling device, the first angling device can be connected to at least one of the upright frame member or the first support arm, and the second angling device can be connected to at least one of the upright frame member and the second support arm, and, with the upright frame member supporting the first light panel and the second support arm supporting the third light panel, is configurable to form an angle between the first light panel and the third light panel so that light transmitted from the first face side intersects light transmitted from the third face side.

In an implementation, the first support arm and the second support arm are on opposite sides of the upright frame member, and the first angling device can include a first hinge rotatably connecting the first support arm to the upright frame member and the second angling device can include a second hinge rotatably connecting the second support arm to the upright frame member.

In an implementation, the base can include: a base hub portion; and a plurality of base members extended from the base hub portion, each base member of the plurality of base members having a proximal end connected to the base hub portion and a distal end extended away from the base hub portion.

In an implementation, the first support arm can have a first support arm length and each base member of the plurality of base members can have a base member length that is greater than or equal to the first support arm length.

In an implementation, the base hub portion can have a rectangular shape.

In one aspect of the present disclosure a light panel support stand can include: a base; an upright frame member connected to the base; a first support arm configured to support a first light panel having a first front face; a second support arm configured to support a second light panel having a second front face; and a hinge rotatably connecting at least one of the first support arm and the second support arm to the upright frame member so that the first support arm is positionable at an angle relative to the second support arm by rotation, relative to the upright frame member, of at least one of the first support arm and the second support arm and light transmitted from the first front face intersects light transmitted from the second front face.

In an implementation, the hinge can include a first hinge rotatably connecting the first support arm to the upright frame member and a second hinge rotatably connecting the second support arm to the upright frame member so that rotation of at least one of the first support arm and the second support arm relative to the upright frame member forms the angle.

In another aspect of the present disclosure a light panel support stand can further include: a first mounting plate attachable to the first support arm to support the first light panel on the first support arm; and a second mounting plate attachable to the second support arm to support the second light panel on the second support arm.

In another aspect of the present disclosure a light panel support stand can further include: a first mounting bracket attachable to the first light panel and having a first engagement configured to engage a first corresponding engagement on the first mounting plate to hold the first light panel on the first mounting plate; and a second mounting bracket attachable to the second light panel and having a second engagement configured to engage a second corresponding engagement on the second mounting plate to hold the second light panel on the second mounting plate.

In another aspect of the present disclosure a light panel support stand can further include: a first mounting bracket attachable to the first light panel and having a first engagement configured to engage a first corresponding engagement on the first support arm to hold the first light panel on the first support arm; and a second mounting bracket attachable to the second light panel and having a second engagement configured to engage a second corresponding engagement on the second support arm to hold the second light panel on the second support arm.

In an implementation, the hinge is connected to the first support arm, the second support arm, and the upright frame member so that rotation of at least one of the first support arm and the second support arm relative to the upright frame member forms the angle.

These aspects of the disclosure are not meant to be exclusive and other features, aspects, and advantages of the present disclosure will be readily apparent to those of ordinary skill in the art when read in conjunction with the following description, appended claims, and accompanying drawings.

Exposure to electromagnetic radiation (EMR) can be part of a healthy lifestyle. For example, specific natural wavelengths of the sunlight spectrum can drive specific cellular mechanisms. For example, exposure to ultraviolet B (UVB) radiation from the sun can support synthesis of vitamin D within human skin from photochemical reactions occurring in the lower layers of the epidermis. Low Level Light Therapy (LLLT) and Photobiomodulation (PBM) can include Red Light Therapy which can operate to improve health and wellness of individuals, including individuals having various health conditions.

Red Light Therapy can include exposure to EMR having wavelengths between about 400 nanometers (nm) to about 1400 nm. For example, Red Light Therapy can include exposure to EMR having wavelengths of about 400 nm to about 1350 nm, or from about 400 nm to about 1300 nm, or from about 400 nm to about 1250 nm, or from about 400 nm to about 1200 nm, or from about 450 nm to about 1400 nm, or from about 500 nm to about 1400 nm, or from about 550 nm to about 1400 nm, or from about 600 nm to about 1400 nm, or from about 450 nm to about 1350 nm, or from about 500 nm to about 1300 nm, or from about 550 nm to about 1250 nm, or from about 600 nm to about 1200 nm. Exposure to EMR in these ranges of wavelengths can penetrate into human skin and stimulate a healthy cellular response.

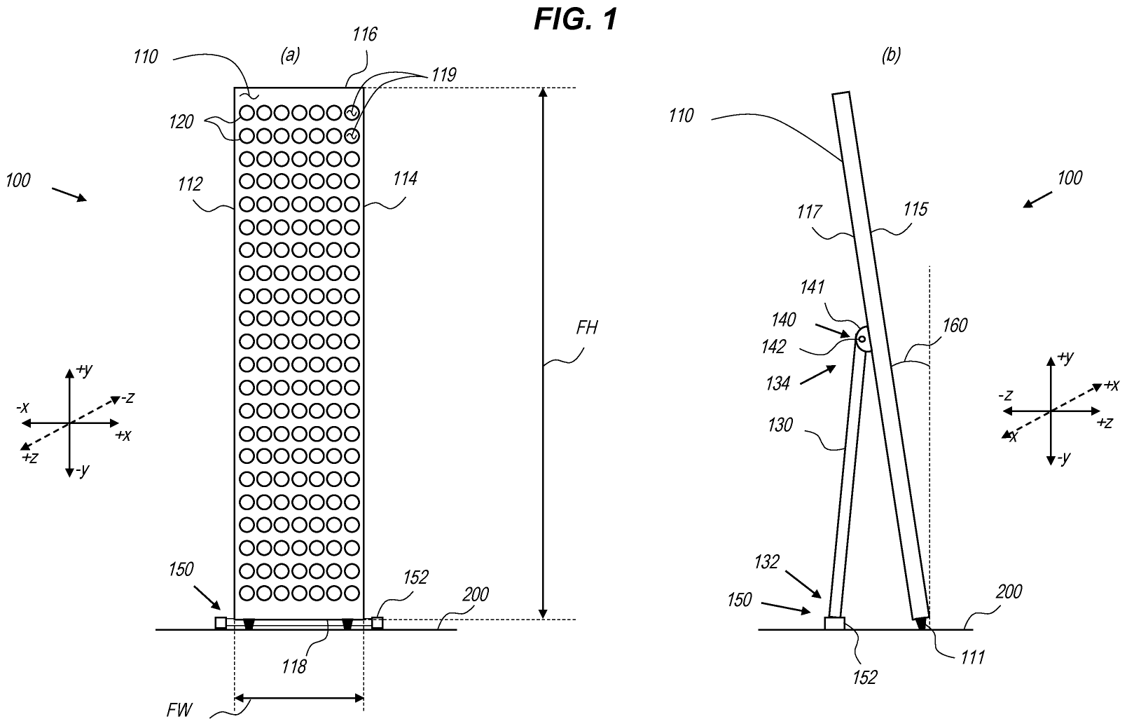

Referring to FIG. 1, the light panel 100 can include a housing 110, a plurality of light emitting diodes 120, a support leg 130, a hinge 140, and a stability bar 150. The light panel 100 can be supported on a floor surface 200 in an upright position (e.g., extending substantially in the +y dimension in the attached figures) by the support leg 130 and the housing 110 (e.g., a bottom side 118 of the housing 110).

The housing 110 can have a face side 115 including a face height FH extending along a major dimension of the housing 110 (e.g., extending substantially along the y-dimension in the attached figures) and a face width FW extending along a minor dimension of the housing 110 (e.g., extending substantially along the x-dimension in the attached figures). The housing 110 can have two sides, a first side 112 and a second side 114, which can be at opposite ends of the face width. The housing 110 can have a top side 116 and a bottom side 118 which can be at opposite ends of the face height FH. The housing 110 can have a depth extending along another minor dimension of the housing 110 (e.g., extending substantially along the z-dimension in the attached figures).

The plurality of light emitting diodes 120 can be inside the housing 110. The plurality of light emitting diodes 120 can be configured to transmit electromagnetic radiation from the face side 115 of the light panel 100. The face side 115 of the housing can include a plurality of openings 119 formed in the face side 115. The plurality of openings 119 can be coincident with the plurality of light emitting diodes 120 so that electromagnetic radiation (e.g., light) emitted by the plurality of light emitting diodes 120 can be transmitted through the plurality of openings 119 from the face side 115 of the housing 110 toward an object to be illuminated. For example, the electromagnetic radiation can be transmitted toward a person seeking to use LLLT (e.g., a user) to improve their health and/or wellness.

The plurality of openings 119 can be spaced in any arrangement along the face side 115 of the housing 110. For example, the plurality of openings 119 can be equally apart along the face side 115 of the housing 110 (e.g., having an equal spacing in at least one of the x-dimension or the y-dimension in the attached figures). In another example, the plurality of openings 119 can be spaced unequally along the face side 115. The plurality of light emitting diodes 120 can be positioned so as to respectively correspond to the plurality of openings 119 so that light emitted by each light emitting diode of the plurality of light emitting diodes 120 is transmitted through the respectively corresponding opening of the plurality of openings 119. The plurality of light emitting diodes 120 can be mounted on a printed circuit board and configured to operate by a controller, driver, or other power supplying device.

The support leg 130 can have a distal end 132 and a proximal end 134 opposite the distal end 132. The proximal end can be rotatably connected to the housing 110 by the hinge 140. The support leg 130 can be rotated along an axis of rotation of the hinge 140 from a closed position, where both the proximal end 134 and the distal end 132 are adjacent the housing 110, to an open position, where the distal end 132 is farther from the housing 110 than the proximal end 134. The hinge 140 can be attached to the housing 110 at the back side 117. The hinge 140 can be attached to the housing 110 at a bottom side 118. The hinge 140 can connect the support leg 130 to the housing 110. The hinge 140 can connect the support leg 130 to the housing 110 at the back side 117, the first side 112, the second side 114, the bottom side 118, or a combination including at least one of the foregoing.

In an implementation, the hinge 140 can connect the support leg 130 to the bottom side 118 so that when the support leg 130 is rotated from the close position to the open position the distal end 132 rotates away from the top side 116 (e.g., rotates from a higher position to a lower position along the y-dimension in the attached figures). The support leg 130 can be contactable with the floor surface 200 along a length of the support leg. In this case, the weight of the light panel 100 can be distributed along a length of the support leg 130 rather than being distributed by the distal end 132 of the support leg 130 and/or stability bar 150 connected thereto.

In the open position, the light panel 100 can be supported on the floor surface 200 in the upright position with a weight of the light panel 100 being distributed between the support leg 130 and the housing 110 (e.g., the bottom side 118 of the housing). The hinge 140 can have an axis of rotation that is substantially horizontal (e.g., substantially parallel to the z-x plane, and the floor surface 200, in the attached figures). The hinge 140 can be attached to the housing 110 along a side surface (e.g., a first side 112, second side 114). In an implementation, the support leg 130 can include two legs, the hinge 140 can be connected on the back side 117 side of the housing 110, and each leg can be attached to opposite sides of the hinge 140. In an implementation, the support leg 130 can include two legs, the hinge 140 can include two hinges (e.g., each attached on opposite sides of the housing 110), and each leg can be attached to one of the two hinges, e.g., a first leg attached to the first side 112 and a second leg attached to the second side 114. The two legs can be independent (e.g., not connected to one another). The two legs can be connected along distal ends 132 (e.g., so as to form a U shape in the x-y plane in the attached figures).

In the closed position the distal end 132 of the support leg can be adjacent to the bottom side 118 of the housing 110. In the open position the distal end 132 of the support leg 130 can be spaced apart from the bottom side 118 of the housing 110. In the open position, the light panel 100 can be supportable on a floor surface 200 by the support leg 130 and the bottom side 118 of the housing 110 in an upright position (e.g., where the major dimension of the light panel 100 is oriented substantially vertically, along the y-dimension in the attached figures). In the open position, the support leg 130 can help prevent the light panel 100 from tipping over (e.g., in the z-dimension in the attached figures). In the open position, the support leg 130 can increase the footprint of the light panel 100 (as described in the following) which can help to increase a minimum tipping force that would need to be applied to tip the light panel 100 over.

The stability bar 150 can help prevent the light panel 100 from tipping over (e.g., in the x-dimension in the attached figures). For example, with the light panel 100 in the upright position (e.g., with the support leg 130 in the open position), the stability bar 150 can increase a minimum tipping force that would need to be applied to tip the light panel 100 over (e.g., in the x-dimension in the attached figures). In an implementation, the stability bar 150 can extend past the first side 112 and/or the second side 114 of the housing 110 thereby increasing the footprint of the light panel 100 (as described in the following) and increasing a minimum tipping force that would need to be applied to tip the light panel 100 over. Furthermore, when the stability bar 150 is weighted, so as to counter-balance the weight of the light panel and shift the center of mass closer to the floor surface 200, the minimum tipping force that would need to be applied to tip the light panel 100 can be further increased.

The support leg 130 can have a length so that the support leg 130 can support the light panel 100 in the upright position when the support leg 130 is in the open position. For example, the support leg can have a length that is greater than or equal to one-half the face height FH of the housing 110. The length of the support leg 130 can be equal to face height FH of the housing 110.

The support leg 130 can be configured so that in the open position the face side 115 of the light panel 100 is tilted at an angle 160 from vertical (e.g., the y-dimension in the attached figures). The angle 160 can include any suitable angle so that the light transmitted from the face side 115 of the light panel 100 is directed toward the skin of a user (e.g., a person seeking LLLT treatment from the light panel 100). The hinge 140 can be configured to allow for the support leg 130 to be adjusted so that the face side 115 of the light panel 100 is set to a desired angle 160. For example, the angle 160 can be from about 5° to about 40°, or from about 5° to about 35°, or from about 10° to about 30°, or from about 10° to about 25°, or from about 15° to about 25°, or from about 15° to about 20°, or about 10°, or about 12°, or about 13°, or about 14°, or about 15°, or about 16°, or about 17°, or about 18°, or about 19°, or about 20°, or about 21°, or about 22°, or about 23° or about 24°, or about 25°, or about 26°, or about 27°, or about 28°, or about 29°, or about 30°. In an implementation, the hinge 140 includes a tightening mechanism to prevent the hinge from rotating so that once the angle 160 is set to the desired angle the tightening mechanism can be tightened to prevent adjustment of the angle 160.

The hinge 140 can be any suitable type of hinging mechanism. For example, the hinge 140 can include a barrel hinge having two leaves, each one attached to one of the rotatable members, one or more knuckles extending from the leaves and connecting the leaves to a pin that provides the axis of rotation. The hinge 140 can include a pin defining the axis of rotation and connecting the rotatable members without leaves or knuckles, so that the rotatable members can rotate relative to the pin. The hinge 140 can include a stopping mechanism to prevent over rotation of one member relative to the other member (e.g., a wedge, hook, or restraint positioned to restrain rotation of the members relative to one another). In an implementation, the hinge 140 can include one or more leaves 141 having a pin hole to accommodate a pin 142 attached to the back side 117 of the housing 110, the proximal end 134 of the support leg 130 can have a corresponding pin hole, and a pin 142 (e.g., determining the axis of rotation of the hinge 140) can extend through the pin hole of the one or more leaves and the pin hole of the support leg 130 to allow for relative rotation between the support leg 130 and the housing 110 about the axis of rotation defined by the pin 142.

In an implementation, the light panel 100 can include a guide attached between the support leg 130 and the housing 110 to guide rotation of the support leg 130 relative to the housing 110. The guide can slide along the housing 110 or the support leg 130 as the support leg is rotated from the housing 110 and can be configured to interact with a protrusion or other projection from either the housing 110 or the support leg 130 to restrain the amount of rotation of the support leg 130 relative to the housing 110.

The distal end 132 of the support leg 130 can be connected to the stability bar 150. The stability bar can extend away from the support leg 130. For example, the stability bar 150 can extend along a minor dimension of the face side 115 of the housing 110 (e.g., extending in the x-dimension in the attached figures). In an implementation, the stability bar 150 can extend beyond the first side 112 in the −x direction and beyond the second side 114 in the +x direction which can widen the stance or footprint of the light panel 100 and improve the stability of the panel so that a force to knock over the light panel (e.g., a force acting in the y-dimension) is higher than without the stability bar 150. In an implementation, the support leg 130 can include two legs that are each attached to the stability bar 150 at their distal end 132 (e.g., the end opposite the hinge 140), thereby connecting the two support legs. In an implementation, the support leg 130 can be U-shaped (e.g., in the x-y plane in the attached figures), the bottom portion of the U shape can be at the distal end 132 of the support leg 130 (e.g., the end opposite the hinge 140), and the bottom portion of the U shape can be attached to the stability bar 150 (e.g., with one or more ties, adhesives, welds, fasteners, such as bolts, screws, and the like, or any other suitable fastening device). In an implementation, the support leg 130 can include a hole at the distal end 132 and the stability bar 150 can be inserted through the hole.

The support leg 130 can operate to increase a footprint of the light panel 100. For example, by extending the distal end 132 of the support leg 130 away from the housing 110 and supporting the light panel 100 in an upright position (e.g., in a substantially vertical position, such that the face height FH extends substantially in the y-dimension in the attached figures) on both the support leg 130 and the bottom side 118 of the housing 110, the footprint of the light panel 100 can be increased beyond the area of the bottom side 118 of the housing 110.

Moreover, the footprint can be increase to multiple times of the area of the bottom side 118 of the housing 110. For example, when the support leg 130 is in the open position, the footprint of the light panel 100 can be increased by about 2 to about 15 times the area of the bottom side 118 of the housing 110, or by about 3 times, or about 4 times, or about 5 times, or about 6 times, or about 7 times, or about 8 times or about 9 times, or about 10 times, or about 11 times, or about 12 times, or about 13, times, or about 14 times, or about 15 times the area of the bottom side 118 of the housing 110. In other words, if the bottom side 118 of the housing 110 has an area of about 50 square inches (in²), then the footprint of the light panel 100 can be increased to about 100 in² to about 750 in² by utilizing a support leg 130 as described herein.

The stability bar 150 can operate to further increase the footprint of the light panel 100. For example, with the stability bar extended beyond the first side 112 and the second side 114 of the housing 110 and the light panel 100 in the upright position, the footprint of the light panel 100 can be increased beyond the area between the of the bottom side 118 and the support leg 130. For example, the footprint can include two triangular sections (e.g., in the x-z plane of the attached figures) extending between either end of the stability bar to the support leg and to the bottom side 118 of the housing 110. In this case, the footprint can be increased by multiples of the footprint when only a support leg 130 is used. For example, when the support leg 130 is in the open position with a stability bar 150, the footprint of the light panel 100 can be increased by about 1.2 to about 4 times the footprint without a stability bar 150. In other words, if the light panel 100 has a footprint of about 500 square inches (in²), then the footprint of the light panel 100 can be increased to about 600 in² to about 2000 in² by utilizing a stability bar 150 as described herein. The increase in footprint can be proportional to distance that the stability bar 150 extends from the support leg 130.

In an implementation, the stability bar 150 can extend away from the support leg 130 beyond the first side 112 and/or the second side 114 of the housing 110 (e.g., in the x-dimension in the attached figures) by an extension amount. The extension amount can be greater than or equal to about one tenth of the face width FW of the light panel 100, for example the extension amount can be greater than or equal to about one fourth of the face width FW of the light panel 100, or greater than or equal to about one third of the face width FW of the light panel 100, or greater than or equal to about one third of the face width FW of the light panel 100, greater than or equal to about one half of the face width FW of the light panel 100. The extension amount can be greater than or equal to about one tenth of the face height FH of the light panel 100, for example the extension amount can be greater than or equal to about one ninth of the face height FH of the light panel 100, or greater than or equal to about one eighth of the face height FH of the light panel 100, or greater than or equal to about one seventh of the face height FH of the light panel 100, greater than or equal to about one sixth of the face height FH of the light panel 100, or greater than or equal to about one fifth of the face height FH of the light panel 100, or greater than or equal to about one fourth of the face height FH of the light panel 100, or greater than or equal to about one third of the face height FH of the light panel 100, or greater than or equal to about one half of the face height FH of the light panel 100. In an implementation, the extension amount can be greater than or equal to about 4 inches, or greater than or equal to about 5 inches, or greater than or equal to about 6 inches, or greater than or equal to about 7 inches, or greater than or equal to about 8 inches, or greater than or equal to about 9 inches, or greater than or equal to about 10 inches.

In an implementation the face height FH of the light panel 100 can be greater than or equal to about 28 inches and the stability bar 150 can have a total length (extended in the x-dimension of the attached figures) of greater than or equal to about 24 inches. For example, the face height FH of the light panel 100 can be greater than or equal to about 30 inches and the stability bar 150 can have a total length of greater than or equal to about 24 inches, or the face height FH of the light panel 100 can be greater than or equal to about 32 inches and the stability bar 150 can have a total length of greater than or equal to about 24 inches, or the face height FH of the light panel 100 can be greater than or equal to about 36 inches and the stability bar 150 can have a total length of greater than or equal to about 24 inches, or the face height FH of the light panel 100 can be greater than or equal to about 40 inches and the stability bar 150 can have a total length of greater than or equal to about 24 inches, or the face height FH of the light panel 100 can be greater than or equal to about 46 inches and the stability bar 150 can have a total length of greater than or equal to about 24 inches.

The stability bar 150 can include a friction surface on a side of the stability bar 150 that contacts the floor surface 200 to reduce the likelihood that the stability bar will slide along the floor surface 200. For example, a friction surface can include a material with a coefficient of static friction of greater than or equal to 0.5, or 0.6, or 0.7, or 0.8, or 0.9, or 1.0 where the coefficient of static friction can be defined as μₛ=Fₘₐₓ/Fₙₒᵣₘₐₗ, the ratio of a maximum force that can be applied to an object before the object starts to move divided by the normal force of the object. For example, the friction surface can include materials such as natural rubber, synthetic rubber, leather, wood, ceramic materials, or other materials having a sufficiently high surface roughness.

The stability bar 150 can include one or more feet 152. For example, the stability bar 150 can include two feet 152, one at each end of the stability bar. The feet 152 of the stability bar 150 can include a friction surface, as previously described, on a side of the feet 152 of the stability bar 150 that contacts the floor surface 200. The feet 152 of the stability bar 150 can be formed from a friction material, e.g., natural or synthetic rubber, leather, wood, ceramic, and the like.

The housing 110, the support leg 130, the stability bar 150, or a combination including at least one of the foregoing can be constructed of a metal (e.g., steel, aluminum, or an alloy thereof), a polymer (e.g., acrylonitrile butadiene styrene (ABS), polyvinyl chloride (PVC), polycarbonate, polypropylene, polyethylene (e.g., high-density polyethylene (HDPE), and the like), or a combination including at least one of the foregoing. The housing 110, the support leg 130, and the stability bar 150 can be constructed to different materials. For example, the housing 110 can be constructed of aluminum (e.g., which can be painted or powder coated) while the support leg 130 and/or stability bar 150 are constructed of steel (e.g., which can be painted or powder coated).

The stability bar 150 can be weighted to counter-balance the light panel 100. For example, the stability bar 150 can be hollow (e.g., having a void space internal to the stability bar 150) and at least partially filled (e.g., at least partially filling the void space) with a weighting material such as sand, water, lead, or other dense material. The stability bar 150 can be solid (e.g., without void space internal to the stability bar 150) and constructed of a dense material such as a metal (e.g., steel, steel alloy, aluminum, aluminum alloy, or the like). For example, a weighted stability bar can lower the center of mass of the light panel 100 by increasing the proportion of the total mass of the light panel 100 that is near the floor surface 200 which can help stabilize the light panel 100 on a floor surface 200. The light panel 100 can weigh up to 30 pounds (lbs.) which can make the light panel 100 prone to tip over if not properly supported. The stability bar 150 can have a weight of between about 5 lbs. to about 50 lbs., or from about 10 lbs. to about 45 lbs., or from about 10 lbs. to about 40 lbs., or from about 15 lbs. to about 40 lbs., or from about 15 lbs. to about 35 lbs., or from about 15 lbs. to about 30 lbs., or from about 20 lbs. to about 30 lbs., or from about 20 lbs. to about 25 lbs., or about 20 lbs., or about 22 lbs., or about 24 lbs., or about 26 lbs., or about 28 lbs., or about 30 lbs., or about 32 lbs., or about 34 lbs., or about 35 lbs., or about 40 lbs.

The housing 110 can include one or more feet 111. For example, the housing 110 can include two feet 111, one toward the first side 112 and the other toward the second side 114 of the housing 110. The feet 111 of the housing 110 can include a friction surface, as previously described, on a side of the feet 111 of the housing 110 that contacts the floor surface 200. The feet 111 of the housing 110 can be formed from a friction material, e.g., natural or synthetic rubber, leather, wood, ceramic, and the like. The feet 111 of the housing 110 can have any shape, e.g., cylindrical, conical, or the shape of a polyhedron having straight or curved edges and sharp vertices (e.g., shape of a discontinuous function) or soft vertices (e.g., shape of a continuous function).

In an implementation, another (e.g., a second) stability bar 150 can be connected to the bottom side 118 of the housing 110. Including another stability bar 150 in this way can further increase the footprint of the light panel 100. For example, the second stability bar 150 can be attached to the back side 117 or the bottom side 118 of the housing 110 and can support the housing forward of the first stability bar 150 (e.g., in front of the first stability bar 150 in the +z direction in the attached figures). In an implementation, the second stability bar 150 can extend from first side 112 and/or from the second side 114 of the housing 110 and can widen (e.g., along the x-dimension in the attached figures) the footprint of a front portion (e.g., corresponding to the bottom side 118) of the light panel 100.

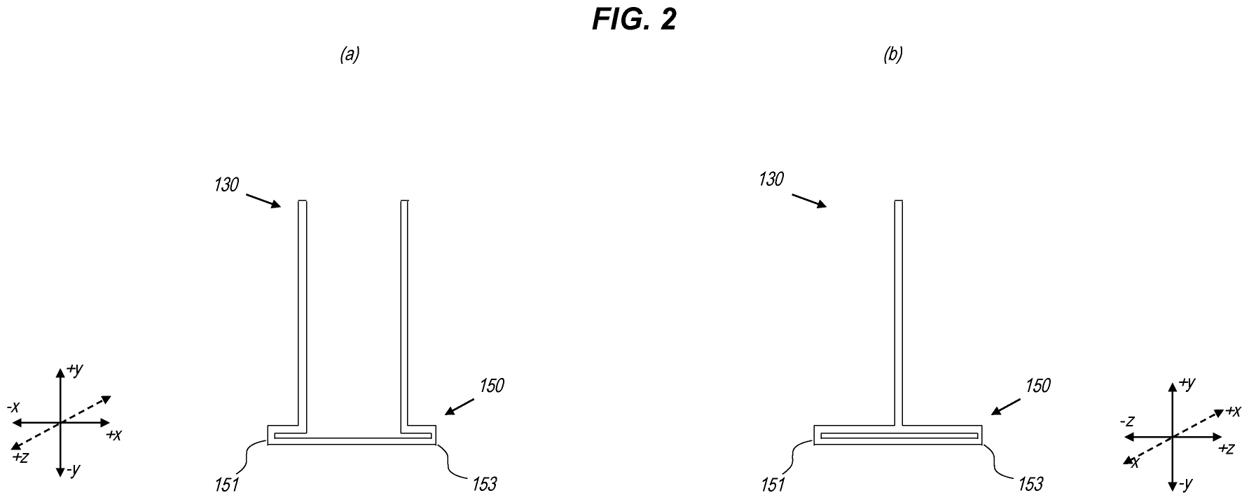

Referring to FIG. 2, the stability bar 150 can be formed integrally with the support leg 130. For example, the support leg can be formed (e.g., molded, bent) into a U shape (e.g., FIG. 2 (a)) or a T shape (e.g., FIG. 2 (b)). In the U shape, the lower portion of the U can form the stability bar 150. In the T shape, the upper portion of the T can form the stability bar 150 and the T shape can be turned upside down when the support leg 130 is rotatably attached to the light panel 100.

The support leg 130 can be formed in the U shape with first extensions 151 and second extension 153 protruding in opposite directions from the lower portion of the U to extend the stability bar 150. For example, the stability bar 150 can include a first extension 151 which can protrude beyond the first side 112 of the housing 110 (e.g., protruding in the −x direction of the attached figures) when the support leg 130 is rotatably attached to the housing 110 and a second extension 153 protruding beyond the second side 114 of the housing 110 (e.g., protruding in the +x direction of the attached figures) when the support leg 130 is rotatably attached to the housing 110.

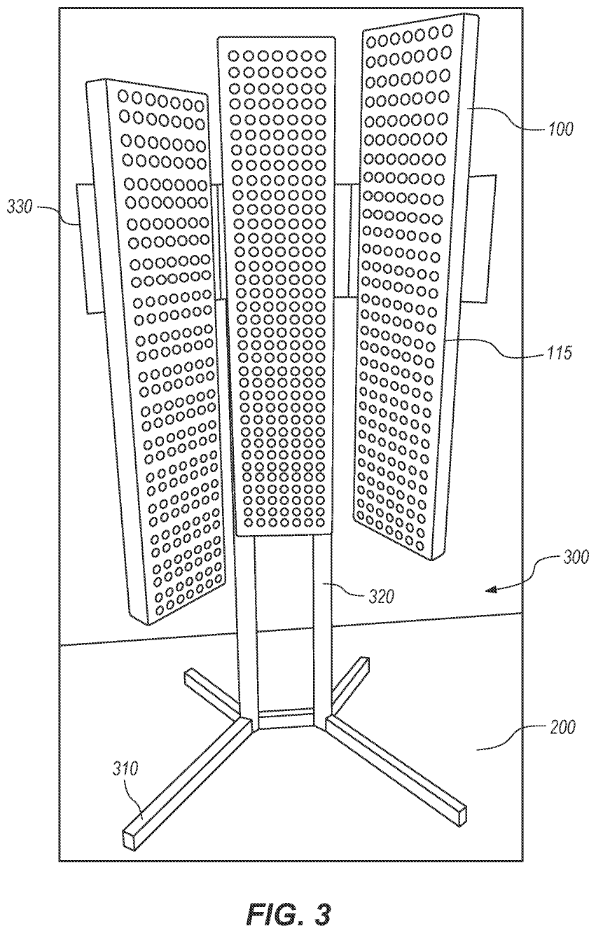

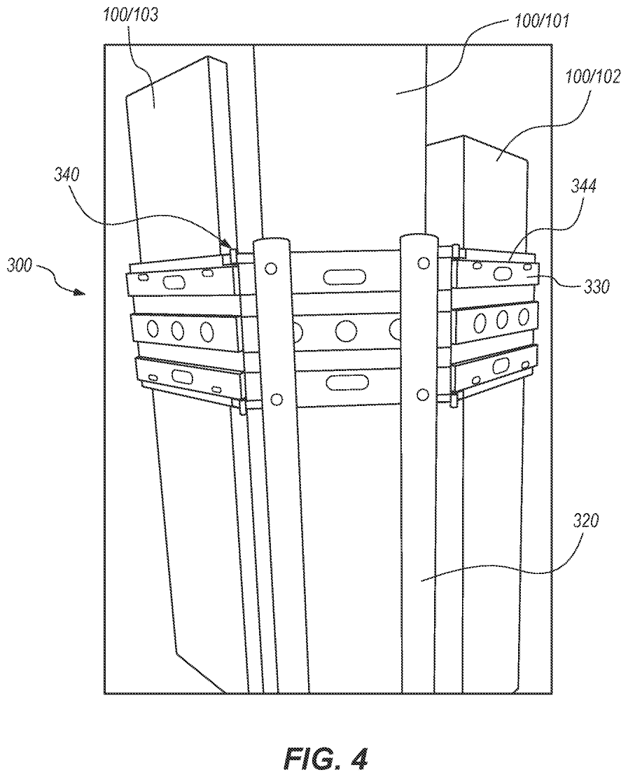

Referring to FIGS. 3-6, a light panel support stand 300 can include a base 310, an upright frame member 320, a support arm 330 and an angling device 340. The light panel support stand 300 can be configured to hold a light panels 100 on the support arm 330 and/or the upright frame member 320.

The base 310 can be configured to sit on a floor surface 200. The base 310 can support the upright frame member 320 in the upright position (e.g., substantially vertical, in the y-dimension of the attached figures). For example, the base 310 can include three or more legs extending from a central portion, or hub, of the base 310. The legs can stabilize the base 310 on the floor surface 200 (e.g., by distributing the weight of the light panel support stand 300 and one or more light panels 100 attached thereto over an area larger than the central portion of the base 310, and/or larger than the bottom side 118 of the one or more light panels 100 attached thereto). The base 310 can include a weight to stabilize the base 310 and/or light panel support stand 300 on the floor surface 200. The weight can lower a center of gravity of the base 310 and/or light panel support stand 300 by providing a greater proportion of the total mass to a lower position thereby biasing the mass distribution in the vertical dimension closer to the floor surface 200. The base 310 can include wheels so that the light panel support stand 300 can be rolled on the floor surface 200. In an implementation, the base 310 of the light panel support stand 300 can include three or more legs extending from a central portion and each leg of the three or more legs can have a wheel attached thereto.

The base 310 of the light panel support stand 300 can be configured to have a footprint which is larger than the footprint of one or more light panels 100 attached thereto. For example, legs of the base 310 can extend from the base hub past one or more outermost sides of the one or more light panels 100 attached to the upright frame member 320 so that the legs extend the extension amount (as previously described). In this way, the base can have a greater footprint (e.g., as in the x-z plane of the attached figures) than the one or more panels supported on the light panel support stand 300.

The base 310 can be configured in a substantially alphabetical (capital or lower case) or numeric shape, such as having a D, E, H, I, K, L, M, N, O, S, t, T, U, V, W, X, Y, 1, 3, 8, or 0 shape, or similar shape (e.g., as in the x-z plane of the attached figures). The base 310 can have a longest dimension of greater than or equal to about 24 inches and a shortest dimension of greater than or equal to about 20 inches, or a longest dimension of greater than or equal to about 26 inches and a shortest dimension of greater than or equal to about 22 inches, or a longest dimension of greater than or equal to about 28 inches and a shortest dimension of greater than or equal to about 24 inches, or a longest dimension of greater than or equal to about 30 inches and a shortest dimension of greater than or equal to about 26 inches, or a longest dimension of greater than or equal to about 32 inches and a shortest dimension of greater than or equal to about 28 inches, or a longest dimension of greater than or equal to about 34 inches and a shortest dimension of greater than or equal to about 30 inches, or a longest dimension of greater than or equal to about 36 inches and a shortest dimension of greater than or equal to about 32 inches, or a longest dimension of greater than or equal to about 38 inches and a shortest dimension of greater than or equal to about 34 inches, or a longest dimension of greater than or equal to about 40 inches and a shortest dimension of greater than or equal to about 36 inches, or a longest dimension of greater than or equal to about 42 inches and a shortest dimension of greater than or equal to about 38 inches, or a longest dimension of greater than or equal to about 44 inches and a shortest dimension of greater than or equal to about 40 inches. The legs of the base 310 can have the same length. The legs of the base 310 can extend the same distance from the hub of the base 310.

The base 310 can have a symmetrical shape (e.g., as in the x-z plane of the attached figures). The base 310 can be symmetrical about the upright frame member 320 (e.g., symmetrical about the x-axis dimension, the y-axis dimension, and/or the z-axis dimension in the attached figures, e.g., with the origin of the Cartesian coordinates is set approximately to where the upright frame member 320 and the base 310 meet).

The base 310 can be configured to be highest (e.g., in the y-dimension in the attached figures) at the hub and lowest (e.g., in the y-dimension in the attached figures) at the distal ends of the legs (e.g., ends opposite to the ends that attach to the hub and/or upright frame member 320). The legs of the base 310 can slope downward from the hub of the base 310 to their distal end. The legs of the base 310 can extend radially from the upright frame member 320 and/or hub of the base 310 and can be spaced circumferentially about the upright frame member 320 and/or hub of the base 310. The legs of the base 310 can extend radially from the upright frame member 320 and/or hub of the base 310 and can be evenly spaced circumferentially about the upright frame member 320 and/or hub of the base 310.

The upright frame member 320 can extend in the vertical direction and can connect the base 310 to the support arm 330. For example, the base 310 and support arm 330 can be welded and/or fastened with mechanical fasteners to the upright frame member 320. The upright frame member 320 can connect a central portion of the base 310 to the support arm 330.

The upright frame member 320 can extend substantially vertically (e.g., in the y-dimension of the attached figures) from the base 310. The upright frame member 320 can include any shape. For example, the upright frame member 320 can include one or more curves between the base and the support arm 330 so as to achieve a particular aesthetic.

The base 310 and/or upright frame member 320 can be formed from elongated members having a cylindrical shape, e.g., formed from hollow bar stock having a round or oval cross-section. The upright frame member 320 can have a polygonal cross-sectional shape, e.g., formed from hollow bar stock having a square, rectangular, triangular, or polygonal cross-section. The upright frame member 320 can have a substantially alphabetical (capital or lower case) or numeric cross-sectional shape, such as including an L, M, N, T, V, X, U, Y, Z, or similar shape, e.g., formed from channel stock, or a bar having any of the aforementioned cross-sectional shapes. The cross-sectional shape of elongated members of the base 310 and the upright frame member 320 can be different.

The base 310 can include a hub section. The hub section can have any shape. The shape of the hub section can be round, oval, triangular, or square, or can be the shape of a polygon having straight or curved sides (e.g., in an x-z plane of the attached figures). In an implementation, the hub section can include an elongated member having a square cross-sectional shape to which a pair of legs and one of two upright frame members 320 are attached at either end.

The upright frame member 320 can be configured to support a light panel 100. For example, the upright frame member 320 can include one or more holes therethrough to allow for a fastener (e.g., bolt, screw, or the like) to attach a light panel 100 to the upright frame member 320 (e.g., by extended the fastener through the housing 110 of the light panel 100 and fastening to the upright frame member 320 or vice-versa). The upright frame member 320 can include a bracket integral, or separate and attached, to the upright frame member 320 which can include one or more holes therethrough to allow for fastening the light panel 100 to the upright frame member 320 (e.g., by extended a fastener through the housing 110 of the light panel 100 and fastening to the bracket of the upright frame member 320 or vice-versa).

The upright frame member 320 or the support arm 330 can include a hanger portion 370 configured to support a hanger extended from the light panel 100. For example, the light panel 100 can include a hanger attached to, and extended from, the back side 117 of the housing 110 and the hanger can be hung onto the hanger portion 370 of the upright frame member 320. The upright frame member 320 can include a vertical spacer 347 for supporting the light panel 100.

The upright frame member 320 can include a front side 323. The front side 323 can correspond to a face side 115 of a light panel 100 when a light panel 100 is attached to the upright frame member 320, or a support arm 330 or bracket 344 attached thereto. The front side 323 can be angled by a first angle 326 relative to a first reference plane RP1. The upright frame member 320, can be configured to directly hold a light panel 100 on the upright frame member 320, e.g., so that the face side 115 is parallel to the front side 323. For example, the upright frame member 320 can include holes for receiving bolts or screws which extend through the light panel 100 to attach the light panel 100 to the upright frame member 320 or vice-versa. The upright frame member 320 can include a hanging portion or hook portion for interacting with a corresponding hanging feature or hook feature of a light panel 100, or vice-versa, so that the light panel 100 can be attached to the upright frame member 320 by interaction between the hanging portion and hanging feature or hook portion and hook feature.

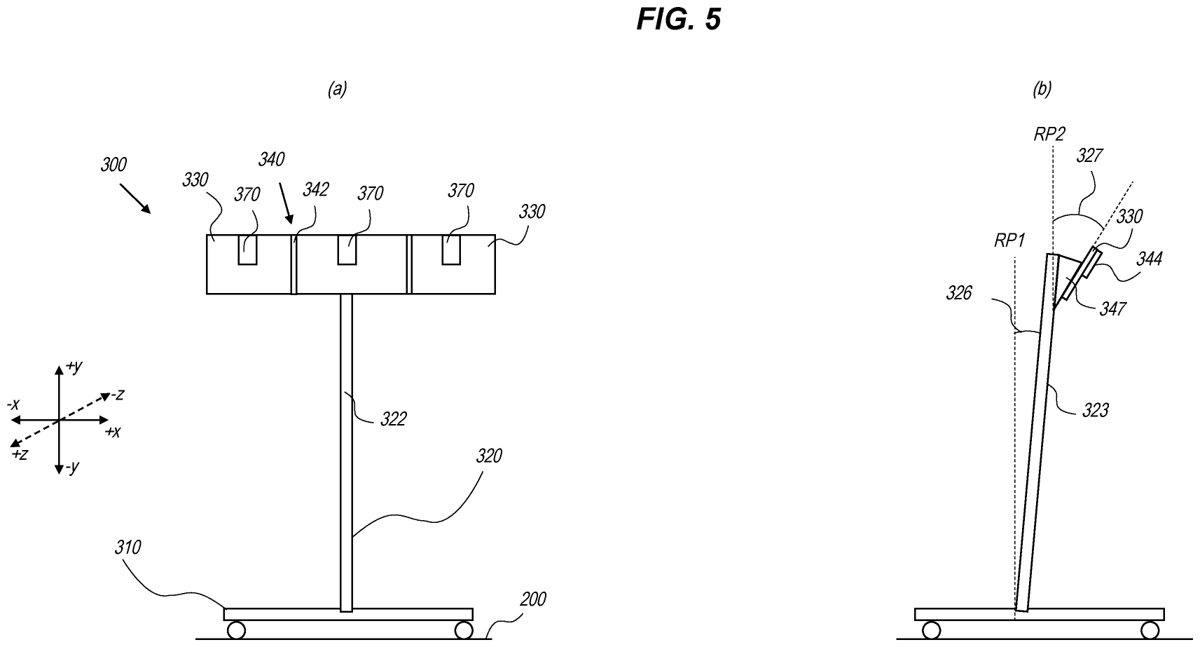

The upright frame member 320, can be configured to support the light panel 100 so that the face side 115 is angled by first angle 326 relative to the first reference plane RP1 and/or angled by second angle 329 relative to a second reference plane RP2. The upright frame member 320 can include a vertical spacer 347 between the upright frame member 320 and the light panel 100 (e.g., so that a second angle 329 is adjustable relative to a second reference plane RP2). The vertical spacer 347 can be replaced by a hinge 140 having an axis of rotation substantially extending in a horizontal dimension such as the x-dimension or z-dimension in the attached figures). Although shown with a triangular cross-section shape in FIG. 5 (b), vertical spacer 347 can have any cross-sectional shape, e.g., including a square or rectangular cross-sectional shape. Adjustment of the first angle 326 and/or second angle 327 can allow for the user of the light panel 100 to set the light panel 100 to a preferred angle for receiving LLLT treatment.

The light panel 100 can be directly mounted onto the upright frame member 320 and can be angled by the first angle 326 relative to the first reference plane RP1 (e.g., a vertical reference plane). The first angle 326 can be set by the upright frame member 320. The light panel 100 can be mounted onto a support arm 330, or a bracket 344 attached thereto, and angled by a second angle 327 relative to the second reference plane RP2. The second reference plane RP2 can extend parallel to the first reference plane RP1 (as shown) or could extend parallel to the upright frame member 320 (e.g., angled by first angle 326 relative to the first reference plane RP1).

The second angle 327 can be set by a hinge 342 or a vertical spacer 347 between the upright frame member 320 and the support arm 330 or a bracket 344 interposed therebetween. The light panel 100 can be connected to the support arm 330 directly (e.g., without a bracket 344 interposed therebetween). The light panel 100 can be connected to the hinge 342 or vertical spacer 347 directly, e.g., so that the hinge 342 or vertical spacer 347 is an intermediate component between the upright frame member 320 and the light panel 100. The light panel 100 can be attached to a bracket 344 which is attached to the support arm 330 so that the bracket 344 is an intermediate component between the support arm 330 and the light panel 100.

The light panel support stand 300 can include a bracket 344 for attaching the one or more light panels 100 on one of the support arm 330 and/or the upright frame member 320. The bracket 344 can be an intermediate component, e.g., between the light panel 100 and the upright frame member 320 or the support arm 330. In an implementation, a bracket 344 can be fixed to the housing 110 of the light panel 100 and the bracket 344 can then be attached on the upright frame member 320 (or extension thereof) or support arm 330 (or extension thereof). In an implementation, the support arm 330 can be coextensive with the bracket 344 so that the bracket 344 and the support arm 330 are one in the same element.

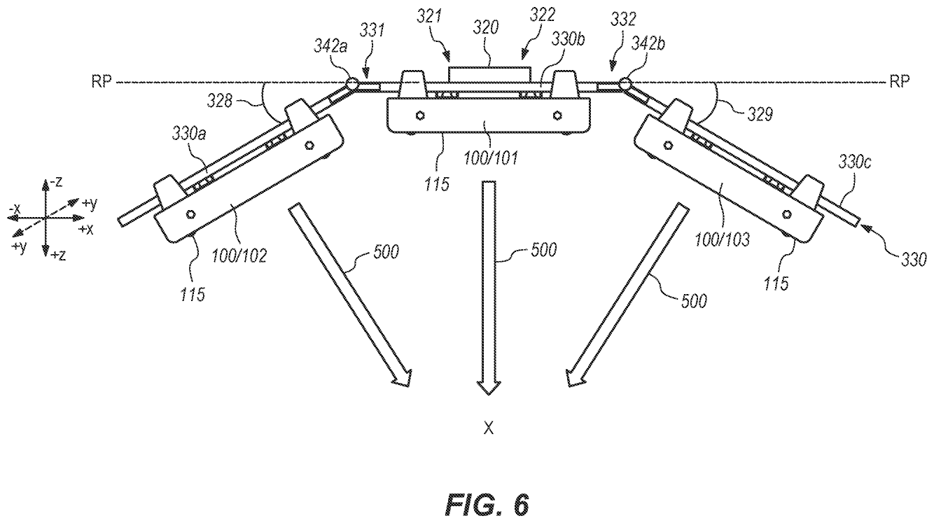

Referring to FIG. 6, a support arm 330 can be configured to attach more than one light panel 100. A support arm 330 can include one or more sections for attaching the one or more light panels 100. For example, the support arm 330 having three sections (330a, 330b, and 330c) connected by a first hinge 342a and a second hinge 342b. The support arm 330 can be used to attach three light panels 100 to the upright frame member 320 of the light panel support stand 300. The first section 330a (e.g., a left section) can be pivotally connected to a first side 331 of a second section 330b (e.g., a middle section) by a first hinge 342a. The third section 320c (e.g., a right section) can be pivotally connected to a second side 332 (e.g., opposite the first side) of the second section 330b. The upright frame member 320 can be attached to any one of the first section 330a, second section 330b, or third section 330c. In an implementation, the upright frame member 320 can be attached to the second section 330b so that the first section 330a extends from a first side 321 of the upright frame member 320 and the third section 330c extends from a second side 322 (e.g., opposite the first side) of the upright frame member 320.

The first hinge 342a can allow for pivotal adjustment to set a first angle 328 between a reference plane RP and the first section 330a. The second hinge 342b can allow for pivotal adjustment to set a second angle 329 between a reference plane RP and the third section 330c. The first angle 328 can be a different angle than the second angle 329. Either of the first angle 328 or the second angle 329 can be angled from about 0° to about 90° relative to the reference plane RP (e.g., which can be defined by the plane of the second section 330b). For example, the first angle 328 and/or the second angle 329 can be from set to an angle from the reference plane RP of about 0° to about 60°, or from about 0° to about 50°, or from about 0° to about 45°, or from about 0° to about 40°, or from about 0° to about 35°, or from about 0° to about 30°, or from about 0° to about 25°, or from about 0° to about 20°, or from about 5° to about 45°, or from about 5° to about 40°, or from about 5° to about 35°, or from about 5° to about 30°, or from about 5° to about 25°, or from about 5° to about 20°, or from about 10° to about 45°, or from about 10° to about 40°, or from about 10° to about 35°, or from about 10° to about 30°, or from about 10° to about 25°, or from about 10° to about 20°, or from about 15° to about 45°, or from about 15° to about 40°, or from about 15° to about 35°, or from about 15° to about 30°.

In this way, when a light panel 100 is attached to each of the three sections (330a, 330b, and 330c), the first angle 328 and the second angle 329 can define a corresponding first angle and a second angle between the three light panels 100. These angles can be adjusted (as previously described) to position the face side 115 of each of the three light panels 100 so that light transmitted from the face side 115 of each of the three light panels 100 intersect. Accordingly, the user of the light panel support stand 300 with a support arm 330 having three sections and having light panels 100 attached thereto, can position the light panels 100 so that light transmitted from the light panels 100 as shown in by arrows 500 can intersect at a target location X (e.g., positioned a selected distance from the light panels 100) where the user can position their body. Accordingly, the user can benefit from having a greater portion of light directed at their body in comparison to a support stand that is unable to angle more than one light panel 100 toward the user. In an implementation, it is contemplated, and within the scope of the disclosure, that the upright frame member 320 can be configured to have hinges 342 directly mounted to the upright frame member 320 in a way to achieve the same features as described in the foregoing without the use of support arm 330 having three sections. For example, the upright frame member 320 could be formed in a shape equivalent to the second section 330b from which hinges could be attached to achieve the previously described features.

The first hinge 342a and/or the second hinge 342b can each include a plurality of hinges 342 distributed in the vertical direction between adjacent section of the support arm 330. The first hinge 342a and/or the second hinge 342b can include two or more hinges distributed between the top and bottom of the support arm 330. For example, first hinge 342a can include two hinges, one between a top end of adjacent sections of the support arm 330 and one between a bottom end of adjacent sections of the support arm 330. In another example, first hinge 342a can include three hinges, one between a top end of adjacent sections of the support arm 330, one between a bottom end of adjacent sections of the support arm 330, and one between a middle portion of adjacent sections of the support arm 330.

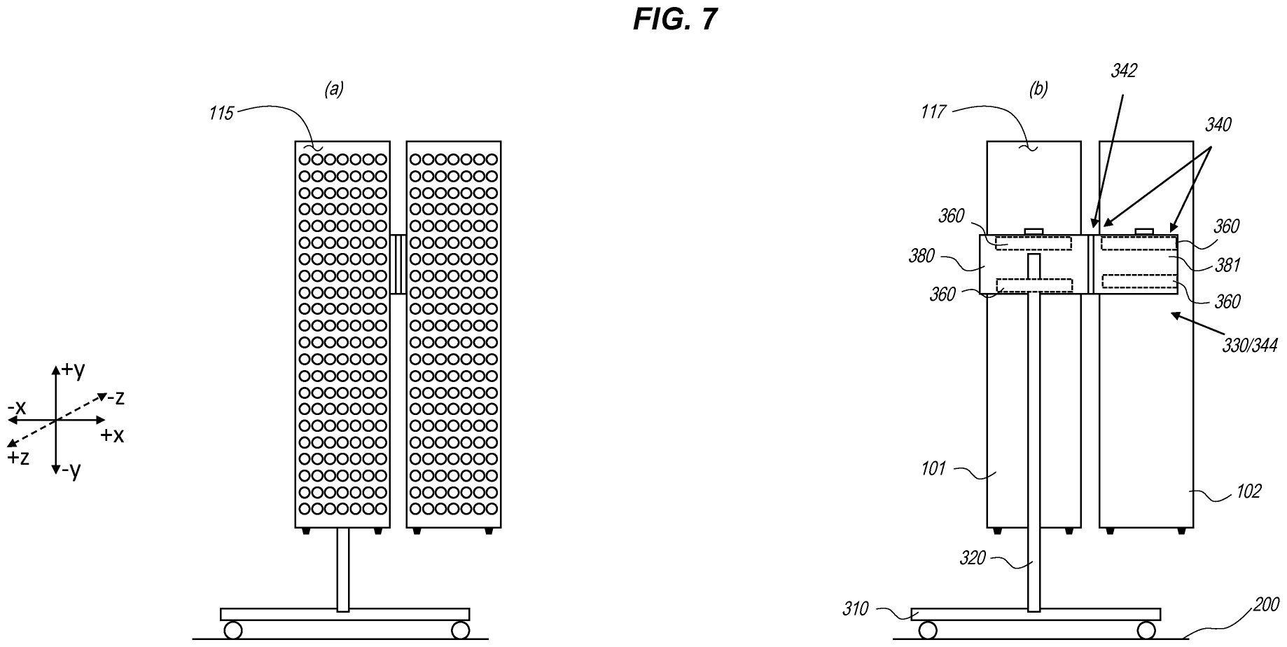

Referring to FIG. 7, the support arm 330 can include two sections. A first section 380 of the two sections can attach to the upright frame member 320 and a second section 381 can be connected to the first section 380 by an angling device 340, such as a hinge 342. A first light panel 101 can be attached to the first section 380 and a second light panel 102 can be attached to the second section 381. When a hinge 342 is used, the hinge 342 can be positioned between the first light panel 101 and the second light panel 102 so that the panels can be pivoted relative to each other without impinging (e.g., so that the housing 110 or each of the two panels doesn't touch until a specific angle between the two panels is reached, e.g., such as less than or equal to about 90°). The hinge 342 can be positioned outboard of the first light panel 101, e.g., at an edge of the first section 380 which is extended beyond a side of the first light panel 101. Alternatively, the two-sections can be attached without a hinge 342, at a fixed angle to one another (e.g., a fixed angle of 90° to 180°). The light panel support stand 300 can include a horizontal spacer 360 to angle the second light panel 102 relative to the first light panel 101, or vice-versa.

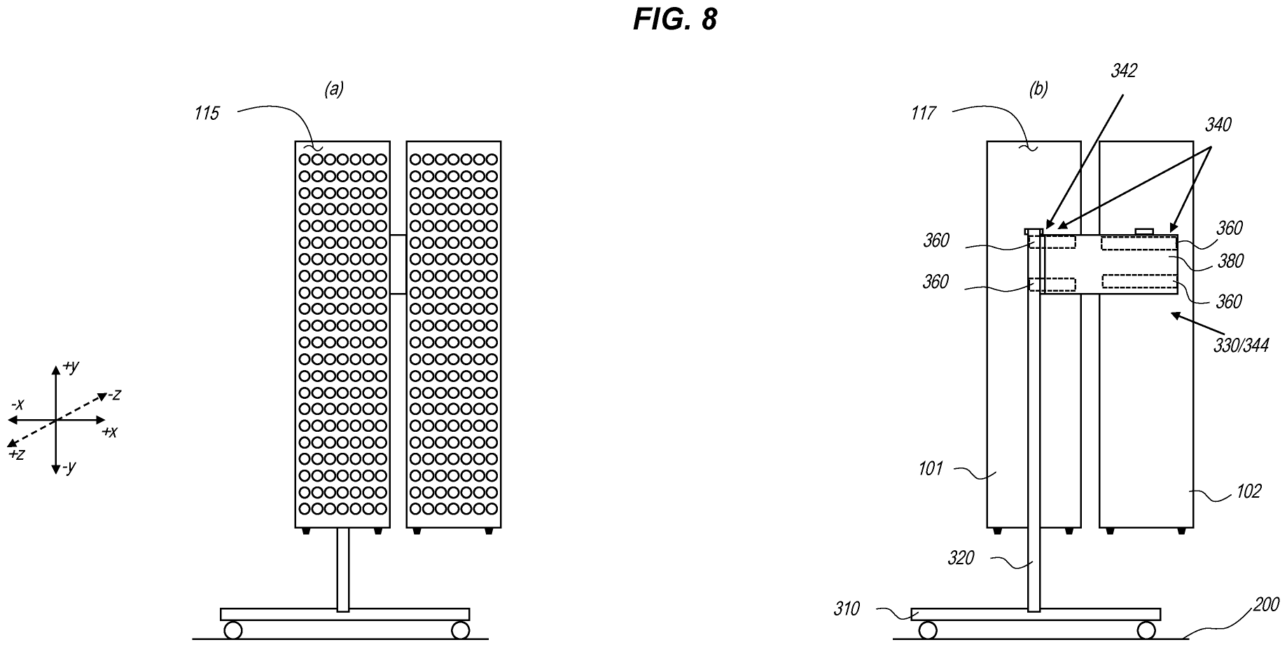

Referring to FIG. 8, the support arm 330 can include a first section 380. The first section 380 can be pivotally connected to the upright frame member 320 by an angling device 340, such as a hinge 342. The first section 380 can be connected to the upright frame member 320 by an angling device 340, such as a horizontal spacer 360. A first light panel 101 can be connected to the upright frame member 320 by an angling device 340, such as horizontal spacer 360, and/or by a bracket 344 so as to be spaced apart from the upright frame member 320 to allow the first section 380 to be angled relative to the first light panel 101 without the first light panel 101 and the second light panel 102 touching. In an implementation, the first light panel 101 is spaced apart from upright frame member 320 with a bracket 344 or horizontal spacer 360 and the first section 380 is pivotally connected to the upright frame member 320 by a hinge 342. In this way, the second light panel 102 can be adjusted relative to the first light panel 101 to allow a user to position the panels to achieve the desired light coverage of a target location.

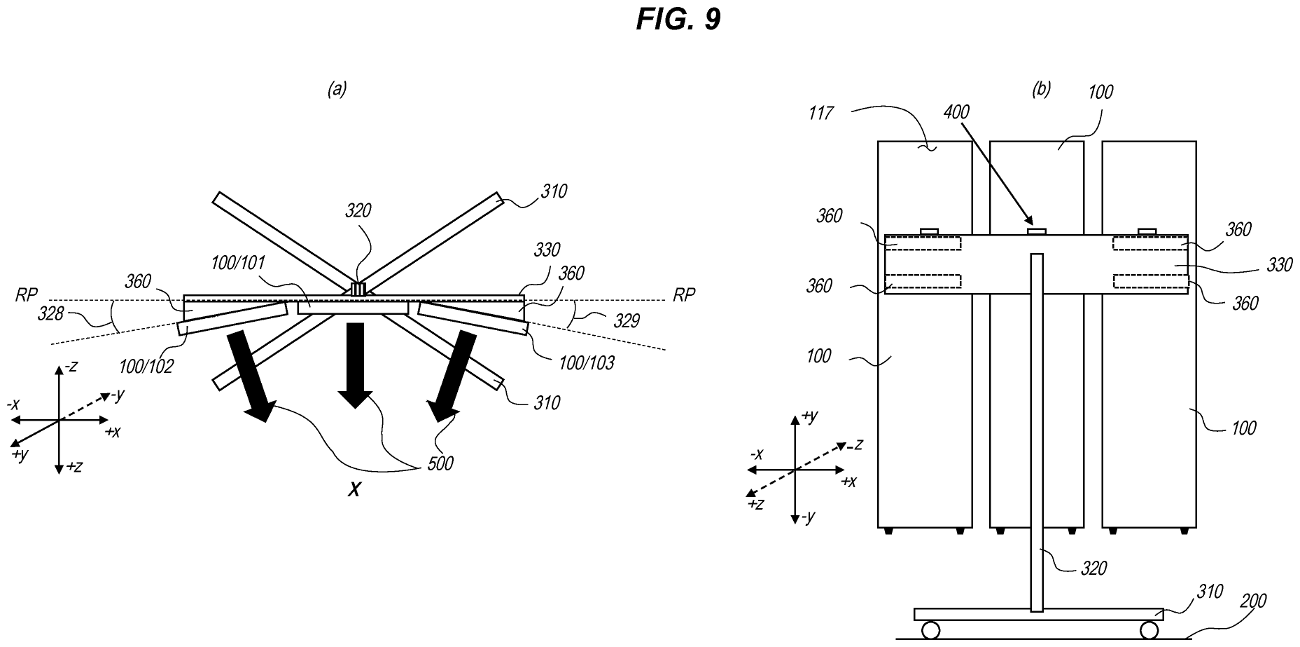

Referring to FIG. 9, the light panel support stand 300 can include a horizontal spacer 360 between the support arm 330 and a light panel 100 attached thereto. The support arm 330 can have multiple sections which extend in a coplanar fashion. The horizontal spacer 360 can be positioned between the light panel 100 and a section of the support arm 330 to angle the light panel 100 toward a target location X. A horizontal spacer 360 can have any cross-sectional shape, e.g., including a square or rectangular cross-sectional shape. As described in the foregoing, adjustment of the first angle 328 and/or second angle 329 can allow for the user of the light panel 100 to set the light panel 100 to a preferred angle for receiving LLLT treatment. The horizontal spacer 360 can be provided in a plurality, such as in sets of two or more for each light panel 100 that is connected thereto. By providing the horizontal spacer 360 in a plurality the light panel 100 attached thereto can be supported firmly when attached to the light panel support stand 300.

Angling device 340 can be connectable to at least one of the upright frame member 320 and the support arm 330. Angling device 340 can be configurable to form an angle between two light panels 100 so that light transmitted from the face side 115 of each of the two light panels 100 intersects. In an implementation, a first light panel 101 can be supported by the upright frame member 320 (e.g., substantially parallel to the first reference plane), the second light panel 102 can be supported by the support arm 330, and angling device 340 (e.g., hinge 342 and/or horizontal spacer 360) can be configured to form an angle between the first light panel 101 and the second light panel 102 so that light transmitted from the face side 115 of the first light panel 101 intersects light transmitted from the face side 115 of the second light panel 102. In an implementation, the first light panel 101 can be supported by a middle section of the support arm 330 (e.g., substantially parallel to the reference plane RP), the second light panel 102 can be supported by left section of the support arm 330, and a third light panel 103 can be supported by a right section of the support arm 330. The angling device 340 can be configured to form an angle between the each of the three light panels 100 so that light transmitted from the face side 115 of the first light panel 101 intersects light transmitted from the face side 115 of the second light panel 102 and light transmitted from the face side 115 of the third light panel 103.

Base 310, upright frame member 320, support arm 330, angling device 340 (e.g., hinge 342 and/or horizontal spacer 360), vertical spacer 347, or a combination including at least one of the foregoing can be can be constructed of a metal (e.g., steel, aluminum, or an alloy thereof), a polymer (e.g., acrylonitrile butadiene styrene (ABS), polyvinyl chloride (PVC), polycarbonate, polypropylene, polyethylene (e.g., high-density polyethylene (HDPE), and the like), wood, or a combination including at least one of the foregoing. In an implementation, base 310, upright frame member 320, and support arm 330 are constructed of a steel alloy. In an implementation, base 310, upright frame member 320, and support arm 330 are constructed of an aluminum alloy. In an implementation, base 310, upright frame member 320, and support arm 330 are constructed of a metal (e.g., such as a steel, aluminum or alloy thereof) which is powder coated. Powder coating can include a pigment to color the element to which the power coating is applied, e.g., base 310, upright frame member 320, support arm 330, or a combination including at least one of the foregoing can be colored with a pigmented powder coat to achieve a particular aesthetic (e.g., selectable by a user).

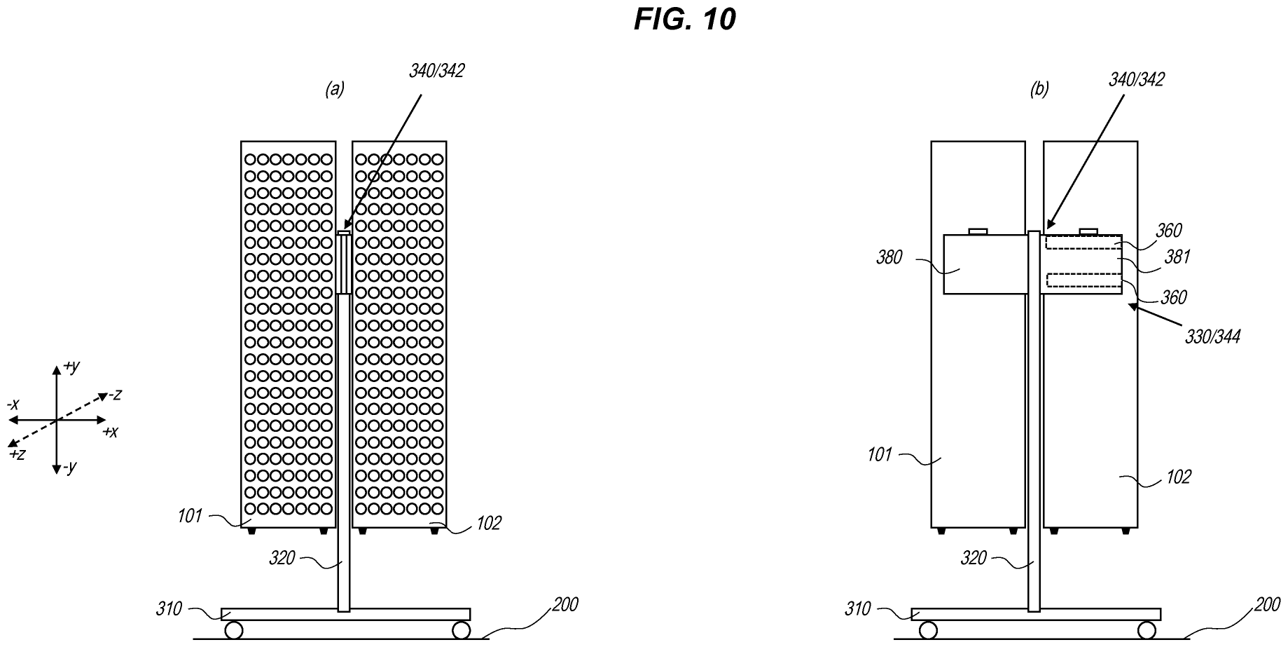

Referring to FIG. 10, the support arm 330 can include a first section 380 and a second section 381. The first section 380 can be connected to the upright frame member 320. The second section can be connected to the upright frame member 320. The first section 380 and the second section 381 can be connected to one another. The first section 380 and/or the second section 381 can be connected to the upright frame member 320. The first section 380 and the second section 381 can be connected to one another and one of the first section 380 or the second section 381 can be connected to the upright frame member 320.

The first section 380 can be pivotally connected to the upright frame member 320 or to the second section 381 by an angling device 340, such as a hinge 342. The second section 381 can be pivotally connected to the upright frame member 320 or to the first section 380 by an angling device 340, such as a hinge 342. A first light panel 101 can be connected to the first section 380 by an angling device 340, such as horizontal spacer 360, and/or by a bracket 344 interposed between the first section 380 and the first light panel 101 so that the first light panel 101 can be spaced apart from the first section 380 and/or to allow the first light panel 101 to be angled relative to the first section 380. A second light panel 102 can be connected to the second section 381 by an angling device 340, such as horizontal spacer 360, and/or by a bracket 344 interposed between the second section 381 and the second light panel 102 so that the second light panel 102 can be spaced apart from the second section 381 and/or to allow the second light panel 102 to be angled relative to the second section 381.

In an implementation, the first section 380 and the second section 381 are each pivotally connected to the upright frame member 320 by an angling device 340 such as a hinge 342. In this way, each of the first section 380 and the second section 381 can be adjusted relative to one another to allow a user to position the panels to achieve the desired light coverage of a target location

In an implementation, the first section 380 and the second section 381 are connected in a fixed way to the upright frame member 320 (e.g., such as welded or bolted). The first section 380 and the second section 381 are fixed at a selected angle to one another that is not adjustable by a user. For example, the first section 380 and the second section 381 can be fixed to the upright frame member 320 so that between the two sections there is a fixed angle of about 90° to about 180°, or about 100° to about 170°, or about 110° to about 160°, or about 110° to about 150°, or about 110° to about 140°, or about 110° to about 135°, or about 110° to about 130°, or about 110° to about 125°, or the like.

In an implementation, the first section 380 is connected in a fixed way to the upright frame member 320 (e.g., such as welded or bolted) and the second section 381 is connected in a pivotable way to the upright frame member 320 (e.g., with a hinge 342). A horizontal spacer 360 can be between the first section 380 and the first light panel 101 to set a first angle 328 between the first section 380 and the first light panel 101. The hinge 342 allows the second section 381 and the second light panel 102, attached thereto, to be adjusted relative to the first light panel 101 to allow a user to position the panels to achieve the desired light coverage of a target location.

In any of the herein disclosed examples, the support arm 330 can be connected to, and can extend substantially parallel to the floor surface 200 from, the upright frame member 320.

In any of the herein disclosed examples, the light panel support stand 300 can include a mounting plate 400. The mounting plate 400 can be attachable to a light panel 100. The mounting plate 400 can be attachable to the upright frame member 320, the support arm 330, and/or a bracket 344 interposed between the light panel 100 and any of the foregoing. The mounting plate 400 can support a light panel 100 on the upright frame member 320. The mounting plate 400 can support a light panel 100 on the support arm 330. The mounting plate 400 can support a light panel 100 on a bracket 344 interposed between the light panel 100 and the upright frame member 320 or the support arm 330.

In any of the herein disclosed examples, the light panel support stand 300 can include a bracket 344 attachable to a light panel 100 and having a first engagement configured to engage a first corresponding engagement on the upright frame member 320 or the support arm 330 to hold the light panel 100 on the upright frame member 320 or the support arm 330. Alternatively, the bracket 344 can be attachable to the upright frame member 320 or the support arm 330 and the first engagement can be configured to engage a first corresponding engagement on the light panel 100 to hold the light panel 100 on the upright frame member 320 or the support arm 330.

In any of the herein disclosed examples, the light panel support stand 300 can include a plurality of mounting plates 400 attachable to the upright frame member 320 or the support arm 330 to support the a plurality of light panels 100 on the light panel support stand 300

In any of the herein disclosed examples, the light panel support stand 300 can include a plurality of brackets 344 attachable to a respectively corresponding plurality of light panels 100. Each bracket 344 of the plurality of brackets 344 can have an engagement configured to engage a corresponding engagement on the upright frame member 320, or the support arm to hold the light panel 100 on the upright frame member 320 or the support arm 330.

In any of the herein disclosed examples, the light panel 100 can have a planar shape corresponding to a planar shape of the housing 110 (e.g. a flat panel, e.g., without curvature in the panel) where the plurality of light emitting diodes 120 are arranged in a planar array (e.g., the plurality of light emitting diodes 120 can be positioned on a common plane, such as attached to a common circuit board within the housing 110).

In any of the herein disclosed examples, the light panel 100 can include a plurality of light emitting diodes 120 respectively corresponding to, and positioned to transmit electromagnetic radiation from, the plurality of openings 119 of the face side 115 of the light panel 100.

In any of the herein disclosed examples, the light panel 100 can include any size. The light panel 100 can be sized corresponding to the size of a user's body. The light panel 100 can have a face side 115, corresponding to a side of the panel from which light is transmitted to provide light therapy to a user. The face height FH of the light panel 100 can be up to about 72 inches and the face width FW can be up to about 48 inches. For example, the panel can have a face height FH of about 70 inches and a face width FW of about 46 inches, or a face height FH of about 68 inches and a face width FW of about 44 inches, or a face height FH of about 66 inches and a face width FW of about 42 inches, or a face height FH of about 64 inches and a face width FW of about 40 inches, or a face height FH of about 62 inches and a face width FW of about 38 inches, or a face height FH of about 60 inches and a face width FW of about 36 inches, or a face height FH of about 58 inches and a face width FW of about 34 inches, or a face height FH of about 56 inches and a face width FW of about 32 inches, or a face height FH of about 54 inches and a face width FW of about 30 inches, or a face height FH of about 52 inches and a face width FW of about 28 inches, or a face height FH of about 50 inches and a face width FW of about 26 inches, or a face height FH of about 48 inches and a face width FW of about 24 inches, or a face height FH of about 46 inches and a face width FW of about 22 inches, or a face height FH of about 44 inches and a face width FW of about 20 inches, or a face height FH of about 42 inches and a face width FW of about 18 inches, or a face height FH of about 40 inches and a face width FW of about 16 inches, or a face height FH of about 32 inches and a face width FW of about 8 inches, or a face height FH of about 30 inches and a face width FW of about 6 inches, or a face height FH of about 40 inches and a face width FW of about 8 inches, or a face height FH of about 40 inches and a face width FW of about 10 inches, or a face height FH of about 40 inches and a face width FW of about 12 inches, or a face height FH of about 40 inches and a face width FW of about 14 inches, or a face height FH of about 42 inches and a face width FW of about 8 inches, or a face height FH of about 42 inches and a face width FW of about 10 inches, or a face height FH of about 42 inches and a face width FW of about 12 inches, or a face height FH of about 42 inches and a face width FW of about 14 inches, or a face height FH of about 44 inches and a face width FW of about 8 inches, or a face height FH of about 44 inches and a face width FW of about 10 inches, or a face height FH of about 44 inches and a face width FW of about 12 inches, or a face height FH of about 44 inches and a face width FW of about 14 inches, or a face height FH of about 34 inches and a face width FW of about 8 inches, or a face height FH of about 34 inches and a face width FW of about 12 inches, or a face height FH of about 34 inches and a face width FW of about 14 inches, or a face height FH of about 36 inches and a face width FW of about 8 inches, or a face height FH of about 36 inches and a face width FW of about 10 inches, or a face height FH of about 36 inches and a face width FW of about 14 inches, or a face height FH of about 38 inches and a face width FW of about 8 inches, or a face height FH of about 38 inches and a face width FW of about 10 inches, or a face height FH of about 38 inches and a face width FW of about 12 inches.

As used herein, a light panel 100 can refer to a body-sized light panel having a face width FW of greater than or equal to about 8 inches, for example, greater than or equal to about 8.5 inches, or greater than or equal to about 8.75 inches, or greater than or equal to about 9 inches. As used herein, a light panel 100 can refer to a body-sized light panel having a face height FH of greater than or equal to about 28 inches, for example greater than or equal to about 30 inches. Furthermore, as used herein, a light panel 100 can refer to a body-sized light panel having greater than or equal to about 180 individual light emitting diodes, for example, greater than or equal to 190 light emitting diodes, or greater than or equal to 200 light emitting diodes, or greater than or equal to 210 light emitting diodes. Still further, as used herein, a light panel 100 can refer to body-sized a light panel having greater than or equal to about 20 rows of light emitting diodes and greater than or equal to about 3 columns of light emitting diodes, for example, greater than or equal to about 22 rows of light emitting diodes and greater than or equal to about 3 columns of light emitting diodes, or greater than or equal to about 24 rows of light emitting diodes and greater than or equal to about 3 columns of light emitting diodes, or greater than or equal to about 26 rows of light emitting diodes and greater than or equal to about 3 columns of light emitting diodes, or greater than or equal to about 28 rows of light emitting diodes and greater than or equal to about 3 columns of light emitting diodes, or greater than or equal to about 30 rows of light emitting diodes and greater than or equal to about 3 columns of light emitting diodes, or greater than or equal to about 20 rows of light emitting diodes and greater than or equal to about 4 columns of light emitting diodes, or greater than or equal to about 22 rows of light emitting diodes and greater than or equal to about 4 columns of light emitting diodes, or greater than or equal to about 24 rows of light emitting diodes and greater than or equal to about 4 columns of light emitting diodes, or greater than or equal to about 26 rows of light emitting diodes and greater than or equal to about 4 columns of light emitting diodes, or greater than or equal to about 28 rows of light emitting diodes and greater than or equal to about 4 columns of light emitting diodes, or greater than or equal to about 30 rows of light emitting diodes and greater than or equal to about 4 columns of light emitting diodes, or greater than or equal to about 20 rows of light emitting diodes and greater than or equal to about 5 columns of light emitting diodes, or greater than or equal to about 22 rows of light emitting diodes and greater than or equal to about 5 columns of light emitting diodes, or greater than or equal to about 24 rows of light emitting diodes and greater than or equal to about 5 columns of light emitting diodes, or greater than or equal to about 26 rows of light emitting diodes and greater than or equal to about 5 columns of light emitting diodes, or greater than or equal to about 28 rows of light emitting diodes and greater than or equal to about 5 columns of light emitting diodes, or greater than or equal to about 30 rows of light emitting diodes and greater than or equal to about 5 columns of light emitting diodes, or greater than or equal to about 20 rows of light emitting diodes and greater than or equal to about 6 columns of light emitting diodes, or greater than or equal to about 22 rows of light emitting diodes and greater than or equal to about 6 columns of light emitting diodes, or greater than or equal to about 24 rows of light emitting diodes and greater than or equal to about 6 columns of light emitting diodes, or greater than or equal to about 26 rows of light emitting diodes and greater than or equal to about 6 columns of light emitting diodes, or greater than or equal to about 28 rows of light emitting diodes and greater than or equal to about 6 columns of light emitting diodes, or greater than or equal to about 30 rows of light emitting diodes and greater than or equal to about 6 columns of light emitting diodes, or greater than or equal to about 20 rows of light emitting diodes and greater than or equal to about 7 columns of light emitting diodes, or greater than or equal to about 22 rows of light emitting diodes and greater than or equal to about 7 columns of light emitting diodes, or greater than or equal to about 24 rows of light emitting diodes and greater than or equal to about 7 columns of light emitting diodes, or greater than or equal to about 26 rows of light emitting diodes and greater than or equal to about 7 columns of light emitting diodes, or greater than or equal to about 28 rows of light emitting diodes and greater than or equal to about 7 columns of light emitting diodes, or greater than or equal to about 30 rows of light emitting diodes and greater than or equal to about 7 columns of light emitting diodes.

The light emitting diodes of a column or row can be staggered, for example, an element of a column can be vertically offset from the corresponding element of an adjacent column and/or an element of a row can be horizontally offset from the corresponding element of an adjacent row, for example, element 1 of column 1 can be vertically offset from element 1 of column 2 and/or element 1 of row 1 can be horizontally offset from element 1 of row 2. In this way the elements in a column or column can appear staggered, e.g., elements in a column can be not aligned vertically and/or elements in a row can be not aligned horizontally.

In any of the herein disclosed examples, the face side 115 of the light panel 100 can have an aspect ratio (e.g., referred to herein as the ratio of the face height FH to face width FW, having the nomenclature of face height FH:face width FW) of from about 8:1 to about 1:1. For example, the light panel 100 can have an aspect ratio of from about 7.5:1 to about 2:1, or from about 7.2:1 to about 2:1, or from about 7.2:1 to about 2.2:1, or from about 7.2:1 to about 2.4:1, or from about 7.2:1 to about 2.6:1, or from about 7.2:1 to about 2.8:1, or from about 7.2:1 to about 3:1, or from about 7:1 to about 2:1, or from about 7:1 to about 2.2:1, or from about 7:1 to about 2.4:1, or from about 7:1 to about 2.6:1, or from about 7:1 to about 2.8:1, or from about 7:1 to about 3:1, or from about 6.8:1 to about 2:1, or from about 6.8:1 to about 2.2:1, or from about 6.8:1 to about 2.4:1, or from about 6.8:1 to about 2.6:1, or from about 6.8:1 to about 2.8:1, or from about 6.8:1 to about 3:1, or from about 6.6:1 to about 2:1, or from about 6.6:1 to about 2.2:1, or from about 6.6:1 to about 2.4:1, or from about 6.6:1 to about 2.6:1, or from about 6.6:1 to about 2.8:1, or from about 6.6:1 to about 3:1, or from about 6.4:1 to about 2:1, or from about 6.4:1 to about 2.2:1, or from about 6.4:1 to about 2.4:1, or from about 6.4:1 to about 2.6:1, or from about 6.4:1 to about 2.8:1, or from about 6.4:1 to about 3:1, or from about 6.2:1 to about 2:1, or from about 6.2:1 to about 2.2:1, or from about 6.2:1 to about 2.4:1, or from about 6.2:1 to about 2.6:1, or from about 6.2:1 to about 2.8:1, or from about 6.2:1 to about 3:1, or from about 6:1 to about 2:1, or from about 6:1 to about 2.2:1, or from about 6:1 to about 2.4:1, or from about 6:1 to about 2.6:1, or from about 6:1 to about 2.8:1, or from about 6:1 to about 3:1, or from about 5.8:1 to about 2:1, or from about 5.8:1 to about 2.2:1, or from about 5.8:1 to about 2.4:1, or from about 5.8:1 to about 2.6:1, or from about 5.8:1 to about 2.8:1, or from about 5.8:1 to about 3:1, or from about 5.6:1 to about 2:1, or from about 5.6:1 to about 2.2:1, or from about 5.6:1 to about 2.4:1, or from about 5.6:1 to about 2.6:1, or from about 5.6:1 to about 2.8:1, or from about 5.6:1 to about 3:1, or from about 5.4:1 to about 2:1, or from about 5.4:1 to about 2.2:1, or from about 5.4:1 to about 2.4:1, or from about 5.4:1 to about 2.6:1, or from about 5.4:1 to about 2.8:1, or from about 5.4:1 to about 3:1, or from about 5.2:1 to about 2:1, or from about 5.2:1 to about 2.2:1, or from about 5.2:1 to about 2.4:1, or from about 5.2:1 to about 2.6:1, or from about 5.2:1 to about 2.8:1, or from about 5.2:1 to about 3:1, or from about 5:1 to about 2:1, or from about 5:1 to about 2.2:1, or from about 5:1 to about 2.4:1, or from about 5:1 to about 2.6:1, or from about 5:1 to about 2.8:1, or from about 5:1 to about 3:1, or from about 4.8:1 to about 2:1, or from about 4.8:1 to about 2.2:1, or from about 4.8:1 to about 2.4:1, or from about 4.8:1 to about 2.6:1, or from about 4.8:1 to about 2.8:1, or from about 4.8:1 to about 3:1, or from about 4.6:1 to about 2:1, or from about 4.6:1 to about 2.2:1, or from about 4.6:1 to about 2.4:1, or from about 4.6:1 to about 2.6:1, or from about 4.6:1 to about 2.8:1, or from about 4.6:1 to about 3:1, or from about 4.4:1 to about 2:1, or from about 4.4:1 to about 2.2:1, or from about 4.4:1 to about 2.4:1, or from about 4.4:1 to about 2.6:1, or from about 4.4:1 to about 2.8:1, or from about 4.4:1 to about 3:1, or from about 4.2:1 to about 2:1, or from about 4.2:1 to about 2.2:1, or from about 4.2:1 to about 2.4:1, or from about 4.2:1 to about 2.6:1, or from about 4.2:1 to about 2.8:1, or from about 4.2:1 to about 3:1, or from about 4:1 to about 2:1, or from about 4:1 to about 2.2:1, or from about 4:1 to about 2.4:1, or from about 4:1 to about 2.6:1, or from about 4:1 to about 2.8:1, or from about 4:1 to about 3:1, or from about 3.8:1 to about 2:1, or from about 3.8:1 to about 2.2:1, or from about 3.8:1 to about 2.4:1, or from about 3.8:1 to about 2.6:1, or from about 3.8:1 to about 2.8:1, or from about 3.8:1 to about 3:1, or from about 3.6:1 to about 2:1, or from about 3.6:1 to about 2.2:1, or from about 3.6:1 to about 2.4:1, or from about 3.6:1 to about 2.6:1, or from about 3.6:1 to about 2.8:1, or from about 3.6:1 to about 3:1, or from about 3.4:1 to about 2:1, or from about 3.4:1 to about 2.2:1, or from about 3.4:1 to about 2.4:1, or from about 3.4:1 to about 2.6:1, or from about 3.4:1 to about 2.8:1, or from about 3.4:1 to about 3:1, or from about 3.2:1 to about 2:1, or from about 3.2:1 to about 2.2:1, or from about 3.2:1 to about 2.4:1, or from about 3.2:1 to about 2.6:1, or from about 3.2:1 to about 2.8:1, or from about 3.2:1 to about 3:1.

In any of the herein disclosed examples, a hinge 140 and/or a hinge 342 can have an angle of rotation which can be limited to prevent under-rotation and/or over-rotation. For example, the hinge 140 and/or a hinge 342 can have an angle of rotation which is limited to rotate between about 0° and about 270°, or about 5° and about 270°, or about 10° and about 270°, or about 15° and about 270°, or about 20° and about 270°, or about 25° and about 270°, or about 30° and about 270°, or about 35° and about 270°, or about 40° and about 270°, or about 45° and about 270°, or about 50° and about 270°, or about 55° and about 270°, or about 60° and about 270°, or about 65° and about 270°, or about 70° and about 270°, or about 75° and about 270°, or about 80° and about 270°, or about 85° and about 270°, or about 90° and about 270°, or about 95° and about 270°, or about 100° and about 270°, or about 105° and about 270°, or about 110° and about 270°, or about 115° and about 270°, or about 120° and about 270°, or about 125° and about 270°, or about 130° and about 270°, or about 135° and about 270°, or about 140° and about 270°, or about 145° and about 270°, or about 150° and about 270°, or about 155° and about 270°, or about 160° and about 270°, or about 165° and about 270°, or about 170° and about 270°, or about 175° and about 270°, or about 180° and about 270°, or about 0° and about 265°, or about 0° and about 260°, or about 0° and about 255°, or about 0° and about 250°, or about 0° and about 245°, or about 0° and about 240°, or about 0° and about 235°, or about 0° and about 230°, or about 0° and about 225°, or about 0° and about 220°, or about 0° and about 215°, or about 0° and about 210°, or about 0° and about 205°, or about 0° and about 200°, or about 0° and about 195°, or about 0° and about 190°, or about 0° and about 185°, or about 0° and about 180°, or about 0° and about 175°, or about 0° and about 170°, or about 0° and about 165°, or about 0° and about 160°, or about 0° and about 155°, or about 0° and about 150°, or about 0° and about 145°, or about 0° and about 140°, or about 0° and about 135°, or about 0° and about 130°, or about 0° and about 125°, or about 0° and about 120°, or about 0° and about 115°, or about 0° and about 110°, or about 0° and about 105°, or about 0° and about 100°, or about 0° and about 95°, or about 0° and about 90°, or about 90° and about 135°, or about 95° and about 135°, or about 100° and about 135°, or about 105° and about 135°, or about 110° and about 135°, or about 115° and about 135°, or about 120° and about 135°, or about 90° and about 130°, or about 90° and about 125°, or about 90° and about 120°, or about 90° and about 115°, or about 90° and about 110°, or about 90° and about 105°, or about 135° and about 180°, or about 135° and about 175°, or about 135° and about 170°, or about 135° and about 165°, or about 135° and about 160°, or about 135° and about 155°, or about 135° and about 150°, or about 135° and about 145°, or about 90° and about 180°, or about 90° and about 175°, or about 90° and about 170°, or about 90° and about 165°, or about 90° and about 160°, or about 90° and about 155°, or about 90° and about 150°, or about 90° and about 145°, or about 90° and about 140°.

In any of the herein disclosed examples, the hinge 140 can be oriented so that the axis of rotation (e.g., the axis about which the hinged elements rotate) is substantially horizontal (e.g., substantially parallel to the ground surface, or floor surface 200, on which a user stands, or e.g., so as to be substantially perpendicular to the major dimension of a user's body). The hinge 342 can be oriented so that the axis of rotation (e.g., the axis about which the hinged elements rotate) is substantially vertical (e.g., substantially orthogonal to the ground surface, or floor surface 200, on which a user stands, or e.g., so as to be substantially parallel to the major dimension of a user's body).

In an implementation, the hinge 140 can be connected to the housing 110 of the light panel 100 (e.g., to the back side 117, or a side surface 112,114 of the housing 110) and oriented so that the axis of rotation is substantially horizontal (e.g., substantially parallel to the ground surface, or floor surface 200, on which a user stands, or e.g., substantially perpendicular to a major dimension of a user's body). Furthermore, the hinge 140 can have an angle of rotation which is limited to rotate between about 0° and about 45°, or about 0° and about 30°, or about 0° and about 29°, or about 0° and about 28°, or about 0° and about 27°, or about 0° and about 26°, or about 0° and about 25°, or about 0° and about 24°, or about 0° and about 23°, or about 0° and about 22°, or about 0° and about 21°, or about 0° and about 20°, or about 0° and about 19°, or about 0° and about 18°, or about 0° and about 17°, or about 0° and about 16°, or about 0° and about 15°, or about 0° and about 14°, or about 0° and about 13°, or about 0° and about 12°, or about 0° and about 11°, or about 0° and about 10°, or about 2° and about 30°, or about 2° and about 29°, or about 2° and about 28°, or about 2° and about 27°, or about 2° and about 26°, or about 2° and about 25°, or about 2° and about 24°, or about 2° and about 23°, or about 2° and about 22°, or about 2° and about 21°, or about 2° and about 20°, or about 2° and about 19°, or about 2° and about 18°, or about 2° and about 17°, or about 2° and about 16°, or about 2° and about 15°, or about 2° and about 14°, or about 2° and about 13°, or about 2° and about 12°, or about 2° and about 11°, or about 2° and about 10°.

In an implementation, the hinge 342 can connect adjacent sections of the support arm 330 and/or a section of the support arm 330 to the upright frame member 320 and oriented so that the axis of rotation is substantially vertical (e.g., substantially orthogonal to the ground surface, or floor surface 200, on which a user stands, or e.g., so as to be substantially parallel to the major dimension of a user's body). Furthermore, the hinge 342 can have an angle of rotation which is limited to rotate between about 90° and about 135°, or about 91° and about 135°, or about 92° and about 135°, or about 93° and about 135°, or about 94° and about 135°, or about 95° and about 135°, or about 96° and about 135°, or about 97° and about 135°, or about 98° and about 135°, or about 99° and about 135°, or about 100° and about 135°, or about 101° and about 135°, or about 102° and about 135°, or about 103° and about 135°, or about 104° and about 135°, or about 105° and about 135°, or about 106° and about 135°, or about 107° and about 135°, or about 108° and about 135°, or about 109° and about 135°, or about 110° and about 135°, or about 111° and about 135°, or about 112° and about 135°, or about 113° and about 135°, or about 114° and about 135°, or about 115° and about 135°.

The various implementations described herein serve as examples of aspects of the disclosure and should not be interpreted as to limit the scope of the disclosed invention.

While various inventive implementations have been described and illustrated herein, those of ordinary skill in the art will readily envision a variety of other means and/or structures for performing the function(s) and/or obtaining the result(s) and/or one or more of the advantages described herein, and each of such variations and/or modifications is deemed to be within the scope of the implementations described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific inventive implementations described herein. It is, therefore, to be understood that the foregoing implementations are presented by way of example only and that, within the scope of the appended claims and equivalents thereto, implementations may be practiced otherwise than as specifically described and claimed. Implementations of the present disclosure are directed to each individual feature, system, article, material, and/or kit described herein. In addition, any combination of two or more such features, systems, articles, materials, and/or kits, if such features, systems, articles, materials, and/or kits, are not mutually inconsistent, is included within the inventive scope of the present disclosure. It is to be understood that a singular form of a noun corresponding to an item may include one or more of the items, unless the relevant context clearly indicates otherwise.

As used herein, each of the phrases “A or B,” “at least one of A and B,” “at least one of A or B,” “A, B or C,”, A, B, and C″, “at least one of A, B and C,” and “at least one of A, B, or C” may include any one of the listed items, or all possible combinations thereof. For example, use of “at least one of” preceding a group of items should be interpreted in a disjunctive way with respect to the group of items, e.g., so that presence of one item of the group meets the meaning of the recitation.

The term “and/or” includes a combination of a plurality of related listed components, or any component among the plurality of related listed components.

Terms such as “first,” “second,” or “first” or “second” may be used simply to distinguish one component from other components, and do not limit the components in other aspects (e.g., importance or order).

Further, terms such as “front”, “rear”, “top”, “bottom”, “side”, “left”, “right”, “upper”, and “lower” used in the present disclosure are defined based on the drawings, and the shape and location of each component are not limited by the terms.

The term “include” or “have” is intended to indicate the presence of a characteristic, number, step, operation, component, part or any combination thereof described in the present document, and the possibility of the presence or addition of one or more other characteristics or numbers, steps, operations, components, parts or any combination thereof is not precluded.

When a component is “connected,” “coupled,” “supported,” or “in contact” with another component, this includes not only cases in which components are directly connected, coupled, supported, or in contact with each other, but also cases in which they are indirectly connected, coupled, supported, or in contact through a third component.

When a component is disposed “on” another component, this includes not only a case in which the component is in contact with another component, but also a case in which still another member is present between the two components.

A term, such as “about” or “substantially,” is used at a corresponding numerical value or used as a meaning close to the numerical value when manufacturing and material tolerances inherent in the stated meaning are presented, and is used to prevent an unconscientious infringer from unfairly using disclosed contents including a numerical value illustrated as being accurate or absolute in order to help understanding of the present disclosure.

What is claimed is:

1. A light panel comprising:
a housing having:
a face side having a face height extending along a major dimension of the housing and a face width extending along a minor dimension of the housing,
a first side and a second side at opposite sides of the face width, and
a top side and a bottom side at opposite ends of the face height;
a plurality of light emitting diodes configured to transmit electromagnetic radiation from the face side;
a support leg having a proximal end and a distal end;
a hinge rotatably connecting the proximal end to the housing; and
a stability bar on the support leg, extending away from the support leg,
wherein the support leg is rotatable between a closed position where both the proximal end and the distal end are adjacent the housing, and an open position where the distal end is farther from the housing than the proximal end and the light panel is supportable on a floor surface in an upright position with a weight of the light panel being distributed between the support leg and the housing.

2. The light panel of claim 1, wherein the housing includes a back side opposite the face side and the hinge is connected to the housing on the back side.

3. The light panel of claim 1, wherein the hinge includes a first hinge and a second hinge, and the first hinge is connected to the first side and the second hinge is connected to the second side.

4. The light panel of claim 3, wherein the support leg includes a first support leg connected to the first hinge and a second support leg connected to the second hinge.

5. The light panel of claim 1, wherein the stability bar includes a friction surface configured to be contactable with the floor surface and prevent the stability bar from sliding on the floor surface with the light panel is in the upright position.

6. The light panel of claim 5, further comprising:
one or more feet connected to the stability bar,
wherein the friction surface includes one or more friction surfaces respectively corresponding to the one or more feet.

7. The light panel of claim 1, wherein the stability bar is fastened to the support leg by one or more fasteners.

8. The light panel of claim 1, wherein the support leg includes two or more portions oppositely extended in the minor dimension beyond the first side and the second side of the housing to form the stability bar integral to the support leg.

9. The light panel of claim 1, wherein the plurality of light emitting diodes are configured to emit electromagnetic radiation having a wavelength of greater than or equal to about 400 nanometers and less than or equal to about 1400 nanometers.

10. The light panel of claim 1, wherein the support leg has a support leg length of greater than or equal to one-half the face height of the light panel.

11. The light panel of claim 1, wherein the support leg includes a plurality of support legs and each support leg of the plurality of support legs has a length of greater than or equal to one-half the face height of the light panel.

12. The light panel of claim 1, wherein the hinge has an axis of rotation that is oriented substantially parallel to the floor surface with the light panel in the upright position.

13. The light panel of claim 1, wherein the stability bar is configured to counter-balance the light panel.

14. The light panel of claim 1, wherein the stability bar includes a first stability bar and a second stability bar,
the first stability bar is on the support leg and extends away from the support leg, and
the second stability bar is connected to the bottom side of the housing, extends in the minor dimension beyond the first side of the housing and the second side of the housing, and includes a friction surface configured to be contactable with the floor surface to prevent the second stability bar from sliding on the floor surface with the light panel is in the upright position.

15. The light panel of claim 1, wherein one or more feet are connected to the bottom side of the housing and are interposed between the bottom side of the housing and the floor surface with the light panel in the upright position.

16. The light panel of claim 1, wherein the hinge connects the support leg on a back side of the housing adjacent to the bottom side of the housing and is configured so that the distal end of the support leg rotates away from the top side of the housing when the support leg is rotated from the closed position to the open position, wherein in the open position the support leg is contactable with the floor surface and the weight of the light panel is distributed along a length of the support leg.

17. A light panel stand comprising:
a base;
an upright frame member connected to the base and configured to support a first light panel having a first face side;
a first support arm connected to the upright frame member, and configured to support a second light panel having a second face side; and
an angling device connected to at least one of the upright frame member or the first support arm, and, with the upright frame member supporting the first light panel and the first support arm supporting the second light panel, the angling device is configurable to form an angle between the first light panel and the second light panel so that light transmitted from the first face side intersects light transmitted from the second face side.

18. The light panel stand of claim 17, wherein the angling device includes a hinge rotatably connecting the first support arm to the upright frame member so that the first support arm is rotatable relative to the upright frame member to form the angle.

19. A light panel stand comprising:
a base;
an upright frame member connected to the base;
a first support arm configured to support a first light panel having a first front face;
a second support arm configured to support a second light panel having a second front face; and
a hinge rotatably connecting at least one of the first support arm or the second support arm to the upright frame member so that the first support arm is positionable at an angle relative to the second support arm by rotation, relative to the upright frame member, of at least one of the first support arm or the second support arm and light transmitted from the first front face intersects light transmitted from the second front face.

20. The light panel stand of claim 19, wherein the hinge includes a first hinge rotatably connecting the first support arm to the upright frame member and a second hinge rotatably connecting the second support arm to the upright frame member so that rotation of at least one of the first support arm and the second support arm relative to the upright frame member forms the angle.
